(19) United States
(12) Patent Application Publication
(10) Pub. No.: US 2007/0270588 A1
(43) Pub. Date: Nov. 22, 2007

(54) Pyrrolopyrimidine Derivatives

(75) Inventors: Francois P. Bischoff, Beerse (BE); Ludo E. Kennis, Beerse (BE); Mirielle Braeken, Beerse (BE); Gaston S. M. Diels, Beerse (BE); Atsuro Nakazato, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: Taisho Pharmaceutical Co., Ltd.

(21) Appl. No.: 10/591,765

(22) PCT Filed: Mar. 4, 2005
(86) PCT No.: PCT/JP05/04266

§ 371 Date: Jul. 11, 2007

(30) Foreign Application Priority Data
Mar. 5, 2004 (JP) 2004-061555

(51) Int. Cl. C07D 487/04

(52) U.S. Cl. 544/280

(57) ABSTRACT

According to the present invention, there is provided an antagonist against CRF receptors which is effective as a therapeutic or prophylactic agent for diseases in which CRF is considered to be involved, such as depression, anxiety, Alzheimer's disease, Parkinson's disease, Huntington's chorea, eating disorder, hypertension, gastro-intestinal diseases, drug dependence, cerebral infarction, cerebral ischemia, cerebral edema, cephalic external wound, inflammation, immunity-related diseases, alpecia, irritable bowel syndrome, sleep disorders, epilepsy, dermatitides, schizophrenia, pain, etc. A pyrrolopyrimidine derivative represented by the following formula [I]: 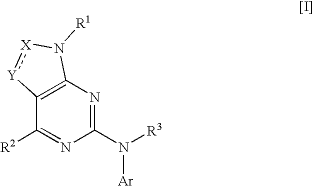 has a high affinity for CRF receptors and is effective against diseases in which CRF is considered to be involved.

### TECHNICAL FIELD

The present invention relates to a therapeutic agent for diseases in which corticotropin releasing factor (CRF) is considered to be involved, such as depression, anxiety, Alzheimer's disease, Parkinson's disease, Huntington's chorea, eating disorder, hypertension, gastro-intestinal diseases, drug dependence, cerebral infarction, cerebral ischemia, cerebral edema, cephalic external wound, inflammation, immunity-related diseases, alpecia, irritable bowel syndrome, sleep disorders, epilepsy, dermatitides, schizophrenia, pain, etc.

### DESCRIPTION OF THE PRIOR ART

CRF is a hormone comprising 41 amino acids (Science, 213, 1394-1397, 1981; and J. Neurosci., 7, 88-100, 1987), and it is suggested that CRF plays a core role in biological reactions against stresses (Cell. Mol. Neurobiol., 14, 579-588, 1994; Endocrinol., 132, 723-728, 1994; and Neuroendocrinol. 61, 445-452, 1995). For CRF, there are the following two paths: a path by which CRF acts on peripheral immune system or sympathetic nervous system through hypothalamus-pituitary-adrenal system, and a path by which CRF functions as a neurotransmitter in central nervous system (in Corticotropin Releasing Factor: Basic and Clinical Studies of a Neuropeptide, pp. 29-52, 1990). Intraventricular administration of CRF to hypophysectomized rats and normal rats causes an anxiety-like symptom in both types of rats (Pharmacol. Rev., 43, 425-473, 1991; and Brain Res. Rev., 15, 71-100, 1990). That is, there are suggested the participation of CRF in hypothalamus-pituitary-adrenal system and the pathway by which CRF functions as a neurotransmitter in central nervous system.

The review by Owens and Nemeroff in 1991 summarizes diseases in which CRF is involved (Pharmacol. Rev., 43, 425-474, 1991). That is, CRF is involved in depression, anxiety, Alzheimer's disease, Parkinson's disease, Huntington's chorea, eating disorder, hypertension, gastrointestinal diseases, drug dependence, inflammation, immunity-related diseases, etc. It has recently been reported that CRF is involved also in epilepsy, cerebral infarction, cerebral ischemia, cerebral edema, and cephalic external -wound (Brain Res. 545, 339-342, 1991; Ann. Neurol. 31, 48-498, 1992; Dev. Brain Res. 91, 245-251, 1996; and Brain Res. 744, 166-170, 1997). Accordingly, antagonists against CRF receptors are useful as therapeutic agents for the diseases described above.

US2004224964 discloses 6,7-dihydro-5H-pyrrolo[2,3-d]pyrimidine derivatives as CRF receptor antagonists. However, none disclose the compounds provided in the present invention.

### PROBLEM(S) TO BE SOLVED BY THE INVENTION

An object of the present invention is to provide an antagonist against CRF receptors which is effective as a therapeutic or prophylactic agent for diseases in which CRF is considered to be involved, such as depression, anxiety, Alzheimer's disease, Parkinson's disease, Huntington's chorea, eating disorder, hypertension, gastro-intestinal diseases, drug dependence, cerebral infarction, cerebral ischemia, cerebral edema, cephalic external wound, inflammation, immunity-related diseases, alpecia, irritable bowel syndrome, sleep disorders, epilepsy, dermatitides, schizophrenia, pain, etc.

### MEANS FOR SOLVING THE PROBLEM

The present inventors earnestly investigated pyrrolopyrimidines that have a high affinity for CRF receptors, whereby the present invention has been accomplished.

The present invention is pyrrolopyrimidine derivatives explained below. A pyrrolopyrimidine derivative represented by the following formula [I]: 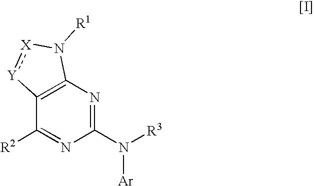
(wherein R¹ is C₁₋₉alkyl, C₂₋₉-alkenyl, C₃₋₇cycloalkyl, C₃₋₇cycloalkyl-C₁₋₉alkyl, di(C₃₋₇cycloalkyl)-C₁₋₉alkyl, C₁₋₆alkoxy-C₁₋₉alkyl, di(C₁₋₆alkoxy)-C₁₋₉alkyl, hydroxy-C₁₋₉alkyl, cyano-C₁₋₉alkyl, carbamoyl-C₁₋₉alkyl, di(C₁₋₆alkyl)amino-C₁₋₉alkyl, aryl, heteroaryl, aryl-C₁₋₉alkyl or heteroaryl-C₁₋₉alkyl, in which said aryl and heteroaryl are optionally substituted with one to three substituents independently selected from the group consisting of C₁₋₆alkyl, C₁₋₆alkoxy, C₁₋₆alkylthio, C₁₋₆alkylsulfonyl, aminosulfonyl, mono(C₁₋₆alkyl)amino sulfonyl, di(C₁₋₆alkyl)aminosulfonyl, halogen, C₁₋₆haloalkyl, cyano, nitro, —NR^{1a}R^{1b}, where R^{1a} and R^{1b} are each independently selected from the group consisting of hydrogen, C₁₋₆alkyl and C₁₋₆alkylcarbonyl;

R² is C₁₋₆alkyl or C₁₋₆haloalkyl;

R³ is hydrogen, C₁₋₆alkyl, C₂₋₆alkenyl, C₂₋₆alkynyl, C₃₋₇cycloalkyl, C₃₋₇cycloalkyl-C₁₋₆alkyl, benzyl;

the bond between X and Y is a single bond or a double bond;

wherein (1) when the bond between X and Y is a single bond, X is CR⁴R⁵ or C═O; Y is CR⁶R⁷, C═O, C═N—OR⁸ or C═CH—R⁹; (2) when the bond between X and Y is a double bond, X is CR¹⁰; Y is CR¹¹;

R⁴ and R⁵ are the same or different, and independently are hydrogen or C₁₋₆alkyl;

R⁶ and R⁷ are the same or different, and independently are hydrogen, C₁₋₆alkyl, C₃₋₆cycloalkyl, C₂₋₆alkenyl, C₂₋₆alkynyl, hydroxy, C₁₋₆alkylamino, di(C₁₋₆alkyl)amino, di(C₁₋₆alkyl)amino-C₁₋₆alkyl, C₁₋₆alkylcarbonylamnino, C₃₋₆cycloalkylcarbonylamino, arylcarbonylamino, heteroarylcarbonylamino, C₁₋₆alkylaminocarbonyl or C₁₋₆alkylaminocarbonylamino; or R⁶ and R⁷ are taken together to form C₃₋₆cycloalkyl, with the proviso that not both of CR⁴R⁵ and CR⁶R⁷ are CH₂;

R⁸ is hydrogen or C₁₋₆alkyl;

R⁹ is C₁₋₆alkyl, C₃₋₆cycloalkyl, aryl or heteroaryl, wherein said aryl and heteroaryl are optionally substituted with one to three substituents independently selected from the group consisting of halogen or C₁₋₆alkyl;

R¹⁰ is hydrogen or C₁₋₆alkyl;

R¹¹ is hydrogen, C₁₋₆alkyl or di(C₁₋₆alkyl)amino-C₁₋₆alkyl;

Ar is aryl or heteroaryl which aryl or heteroaryl is unsubstituted or substituted with 1 or more substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, cyano, C₁₋₆haloalkyl, trifluoromethoxy, difluoromethoxy, fluoromethoxy and —N(R¹²)R¹³, wherein R¹² and R¹³ are the same or different, and independently are hydrogen or C₁₋₆alkyl), individual isomers thereof or racemic or non-racemic mixtures of isomers thereof, or pharmaceutically acceptable salts and hydrates thereof.

The terms used in the present specification have the following meanings.

The term “C₁₋₉alkyl” means a straight chain or branched chain alkyl group of 1 to 9 carbon atoms, such as methyl, ethyl, propyl, isopropyl, butyl, isobutyl, tert-butyl, sec-butyl, pentyl, isopentyl, 1-methylbutyl, hexyl, isohexyl, 1-ethylpropyl, 1-ethylbutyl, 1,3-dimethylbutyl, 1-propylbutyl, 1-propylpentyl, 1-butylpentyl or the like.

The term “C₂₋₉alkenyl” means a straight chain or branched chain alkenyl group of 2 to 9 carbon atoms, such as vinyl, isopropenyl, allyl or the like.

The term “C₃₋₇cycloalkyl” means a cyclic alkyl group of 3 to 7 carbon atoms, such as cyclopropyl, cyclobutyl, cyclopentyl, cyclohexyl, cycloheptyl or the like.

The term “C₃₋₇cycloalkyl-C₁₋₉alkyl” means a substituted C₁₋₉alkyl group having the above-mentioned C₃₋₇cycloalkyl as the substituent, such as cyclopropylmethyl, 1-cyclopropylethyl, 1-cyclobutylethyl, 1-cyclopentylethyl, 2-cyclopropylethyl, 2-cyclobutylethyl, 2-cyclopentylethyl, 1-cyclopropylpropyl, 1-cyclobutylpropyl, 1-cyclopentylpropyl, 1-cyclopropylmethylpropyl, 1-cyclopropylmethylbutyl or the like.

The term “di(C₃₋₇cycloalkyl)-C₁₋₉alkyl” means a substituted C₁₋₉alkyl group having two above-mentioned C₃₋₇cycloalkyl groups as the substituents, such as di(cyclopropyl)methyl, di(cyclobutyl)methyl, di(cyclopentyl)methyl or the like.

The term “C₁₋₆alkoxy” means a straight chain or branched chain alkoxy group of 1 to 6 carbon atoms, such as methoxy, ethoxy, propoxy, isopropyloxy, butoxy, isobutyloxy, pentyloxy, isopentyloxy or the like.

The term “C₁₋₆alkoxy-C₁₋₉alkyl” means a substituted C₁₋₉alkyl group having the above-mentioned C₁₋₆alkoxy group as the substituent, such as methoxymethyl, 2-methoxyethyl, 2-ethoxyethyl, 1-methoxymethyl-propyl, 1-methoxymethyl-butyl or the like.

The term “di(C₁₋₆alkoxy)-C₁₋₉alkyl” means a substituted C₁₋₉alkyl group having two above-mentioned C₁₋₆alkoxy groups as the substituents, such as 2,3-di(methoxy)propyl, 2-methoxy-1-methoxymethyl-ethyl, 2,4-(diethoxy)pentyl or the like.

The term “hydroxy-C₁₋₉alkyl” means a substituted C₁₋₉alkyl group having a hydroxy group, such as hydroxymethyl, 1-hydroxyethyl, 2-hydroxyethyl, 1-hydroxypropyl, 2-hydroxypropyl, 3-hydroxypropyl, 4-hydroxybutyl, 5-hydroxypentyl, 1-hydroxymethyl-propyl, 1-hydroxymethyl-butyl, 1-hydroxymethyl-3-methyl-butyl or the like.

The term “cyano-C₁₋₉alkyl” means a substituted C₁₋₉alkyl group having a cyano group, such as cyanomethyl, 1-cyanoethyl, 2-cyanoethyl, 1-cyanopropyl, 1-cyanobutyl, 5-cyanopentyl, 2-cyano-1-ethyl-ethyl, 1-cyanomethyl-butyl, 1-cyano-3-methyl-butyl, 1-cyanomethyl-3-methyl-butyl or the like.

The term “carbamoyl-C₁₋₉alkyl” means a substituted C₁₋₉alkyl group having a carbamoyl group, such as carbamoylmethyl, 1-carbamoylethyl, 2-carbamoylethyl, 1-carbamoylpropyl, 1-carbamoylbutyl, 5-carbamoylpentyl, 1-carbamoyl-3-methyl-butyl, 1-carbamoylmethyl-butyl, 1-carbamoylmethyl-propyl, 1-carbamoylmethyl-3-methyl-butyl or the like.

The term “di(C₁₋₆alkyl)amino” means an amino group having two above-mentioned C₁₋₆alkyl groups, such as dimethylamino, diethylamino, dipropylamino or the like.

The term “di(C₁₋₆alkyl)amino-C₁₋₉alkyl” means a substituted C₁₋₉alkyl group having an above-mentioned di(C₁₋₆alkyl)amino group, such as 2-dimethylaminoethyl, 3-dimethylaminopropyl or the like.

The term “aryl” means a monocyclic or bicyclic group of 6 to 12 ring carbon atoms having at least one aromatic ring, such as phenyl, naphthyl, or the like.

The term “heteroaryl” means a monocyclic or bicyclic group of 5 to 12 ring atoms having at least one aromatic ring having in its ring 1 to 4 atoms which may be the same or different and are selected from nitrogen, oxygen and sulfur, such as pyridyl, pyrimidinyl, imidazolyl, furyl, thienyl, quinolyl, indolyl, benzofuranyl, quinoxalinyl, benzo[1,2,5]thiadiazolyl, benzo[1,2,5]oxadiazolyl or the like.

The term “aryl-C₁₋₉alkyl” means a substituted C₁₋₉alkyl group having an above-mentioned aryl group, such as benzyl, phenethyl, 3-phenylpropyl or the like.

The term “heteroaryl-C₁₋₉alkyl” means a substituted C₁₋₉alkyl group having an above-mentioned heteroaryl group, such as pyridin-2-ylmethyl, pyridin-3-ylmethyl, pyridin-4-ylmethyl or the like.

The term “C₁₋₆alkylthio” means a straight chain or branched chain alkylthio group of 1 to 6 carbon atoms, such as methylthio, ethylthio, propylthio or the like.

The term “C₁₋₆alkylsulfonyl” means a straight chain or branched chain alkylsulfonyl group of 1 to 6 carbon atoms, such as methylsulfonyl, ethylsulfonyl, propylsulfonyl or the like.

The term “mono(C₁₋₆alkyl)aminosulfonyl” means a substituted aminosulfonyl group having an above mentioned C₁₋₆alkyl, such as methylaminosulfonyl, ethylaminosulfonyl or the like.

The term “di(C₁₋₆alkyl)aminosulfonyl” means a substituted aminosulfonyl group having two above mentioned C₁₋₆alkyl, such as dimethylaminosulfonyl, diethylaminosulfonyl or the like.

The term “halogen” means fluorine, chlorine, bromine or iodine atom.

The term “C₁₋₆haloalkyl” means a substituted C₁₋₆alkyl having one to three halogen atoms, such as trifluoromethyl, difluoromethyl, fluoromethyl, trichloromethyl or the like.

The term “C₁₋₆alkylcarbonyl” means an acyl group of 1 to 7 carbon atoms acetyl, propionyl, butyryl or the like.

The term “C₂₋₆alkynyl” means a straight chain or branched chain alkynyl group of 2 to 6 carbon atoms, such as ethynyl, prop-1-ynyl, prop-2-ynyl or the like.

The term “C₁₋₆alkylamino” means a substituted amino group having an above-mentioned C₁₋₆alkyl group, such as methylamino, ethylamino, propylamino or the like.

The term “C₁₋₆alkylcarbonylamino” means a substituted amino group having a C₁₋₆alkylcarbonyl group, such as acetylamino, propionylamino, 3-methylbutyrylamino, isobutyrylamino, n-butyrylamino or the like.

The term “C₃₋₆cycloalkylcarbonylamino” means a substituted amino group having a C₃₋₆cycloalkylcarbonyl group, such as cyclopropanecarbonylamino, cyclobutanecarbonylamino, cyclopentanecarbonylamino or the like.

The term “arylcarbonylamino” means a substituted amino group having an above mentioned aryl group, such as phenylcarbonylamino or the like.

The term “heteroarylcarbonylamino” means a substituted amino group having an above mentioned heteroaryl group, such as (furan-2-carbonyl)amino, (pyridine-2-carbonyl)amino, (pyridine-3-carbonyl)amino, (pyridine-4-carbonyl)amino or the like.

The term “C₁₋₆alkylaminocarbonyl” means a substituted aminocarbonyl group having an above mentioned C₁₋₆alkyl group, such as methylcarbamoyl, ethylcarbamoyl, isopropylcarbamoyl or the like.

The term “C₁₋₆alkylaminocarbonylamino” means a substituted aminocarbonylamino group having an above mentioned C₁₋₆alkyl group, such as 3-methylureido, 3-ethylureido, 3-propylureido, 3-isopropylureido or the like.

The phrase “aryl or heteroaryl which aryl or heteroaryl is unsubstituted or substituted with 1 or more substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, cyano, C₁₋₆haloalkyl, trifluoromethoxy, difluoromethoxy, fluoromethoxy and —N(R¹²)R¹³, wherein R¹² and R¹³ are the same or different, and independently are hydrogen or C₁₋₆alkyl” includes, for example, 2,4-dimethylphenyl, 2,6-dimethylphenyl, 2,4-dibromophenyl, 2-bromo-4-isoproylphenyl, 2,4-dichlorophenyl, 2,6-dichlorophenyl, 2-chloro-4-trifluoromethylphenyl, 4-methoxy-2-methylphenyl, 2-chloro-4-trifluoromethoxyphenyl, 4-isopropyl-2-methylthiophenyl, 2,4,6-trimethylphenyl, 4-bromo-2,6-dimethylphenyl, 4-bromo-2,6-diethylphenyl, 4-chloro-2,6-dimethylphenyl, 2,4,6-tribromophenyl, 2,4,5-tribromophenyl, 2,4,6-trichlorophenyl, 2,4,5-trichlorophenyl, 4-bromo-2,6-dichlorophenyl, 6-chloro-2,4-dibromophenyl, 2,4-dibromo-6-fluorophenyl, 2,4-dibromo-6-methylphenyl, 2,4-dibromo-6-methoxyphenyl, 2,4-dibromo-6-methylthiophenyl, 2,6-dibromo-4-isopropylphenyl, 2,6-dibromo-4-trifluoromethylphenyl, 2-bromo-4-trifluoromethylphenyl, 4-bromo-2-chlorophenyl, 2-bromo-4-chlorophenyl, 4-bromo-2-methylphenyl, 4-chloro-2-methylphenyl, 2,4-dimethoxyphenyl, 2,6-dimethyl-4-methoxyphenyl, 4-chloro-2,6-dibromophenyl, 4-bromo-2,6-difluorophenyl, 2,6-dichloro-4-trifluoromethylphenyl, 2,6-dichloro-4-trifluoromethoxyphenyl, 2,6-dibromo-4-trifluoromethoxyphenyl, 2-chloro-4,6-dimethylphenyl, 2-bromo-4,6-dimethoxyphenyl, 2-bromo-4-isopropyl-6-methoxyphenyl, 2,4-dimethoxy-6-methylphenyl, 6-dimethylamino-4-methylpyridin-3-yl, 2-chloro-6-trifluoromethylpyridin-3-yl, 2-chloro-6-trifluoromethoxypyridin-3-yl, 2-chloro-6-methoxypyridin-3-yl, 6-methoxy-2-trifluoromethylpyridin-3-yl, 2-chloro-6-difluoromethylpyridin-3-yl, 6-methoxy-2-methylpyridin-3-yl, 2,6-dimethoxypyridin-3-yl, 4,6-dimethyl-2-trifluoromethylpyrimidin-5-yl, 2-dimethylamino-6-methylpyridin-3-yl.

The “pharmaceutically acceptable salts” in the present invention include, for example, salts with an inorganic acid such as sulfuric acid, hydrochloric acid, hydrobromic acid, phosphoric acid, nitric acid or the like; salts with an organic acid such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid, naphthalene-2-sulfonic acid or the like; salts with one or more metal ions such as lithium ion, sodium ion, potassium ion, calcium ion, magnesium ion, zinc ion, aluminium ion or the like; salts with an amine such as ammonia, arginine, lysine, piperazine, choline, diethylamine, 4-phenylcyclohexylamine, 2-aminoethanol, benzathine or the like.

In a compound of the present invention, isomers such as diastereomers, enantiomers, geometric isomers and tautomeric forms may exist. The compound of the present invention includes the individual isomers and the racemic and non-racemic mixtures of the isomers.

Preferable examples of the compound of the present invention are as follows.

The pyrrolopyrimidine derivative represented by the following formula [II]: 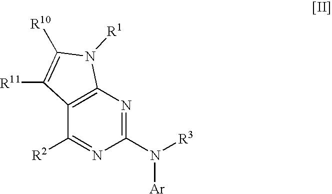
(wherein R¹ is C₁₋₉alkyl, C₂₋₉alkenyl, C₃₋₇cycloalkyl, C₃₋₇cycloalkyl-C₁₋₉alkyl, di(C₃₋₇cycloalkyl)-C₁₋₉alkyl, C₁₋₆alkoxy-C₁₋₉alkyl, di(C₁₋₆alkoxy)-C₁₋₉alkyl, hydroxy-C₁₋₉alkyl, cyano-C₁₋₉alkyl, carbamoyl-C₁₋₉alkyl, di(C₁₋₆alkyl)amino-C₁₋₉alkyl, aryl, heteroaryl, aryl-C₁₋₉alkyl or heteroaryl-C₁₋₉alkyl, in which said aryl and heteroaryl optionally substituted with one to three substituents independently selected from the group consisting of C₁₋₆alkyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, halogen, C₁₋₆haloalkyl, cyano, nitro, —NR^{1a}R^{1b}, where R^{1a} and R^{1b} are each independently selected from the group consisting of hydrogen, C₁₋₆alkyl and C₁₋₆alkylcarbonyl;

R² is C₁₋₆alkyl or C₁₋₆haloalkyl;

R³ is hydrogen, C₁₋₆alkyl, C₂₋₆alkenyl, C₂₋₆alkynyl, C₃₋₇cycloalkyl, C₃₋₇cycloalkyl-C₁₋₆alkyl, benzyl;

R¹⁰ is hydrogen or C₁₋₆alkyl;

R¹¹ is hydrogen, C₁₋₆alkyl or di(C₁₋₆alkyl)amino-C₁₋₆alkyl;

Ar is aryl or heteroaryl which aryl or heteroaryl is unsubstituted or substituted with 1 or more substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, cyano, haloC₁₋₆alkyl, trifluoromethoxy, difluoromethoxy, fluoromethoxy and —N(R¹²)R¹³, wherein R¹² and R¹³ are the same or different, and independently are hydrogen or C₁₋₆alkyl).

More preferable are tho compound represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl, hydroxy-C₁₋₆alkyl, cyano-C₁₋₆alkyl, carbamoyl-C₁₋₆alkyl, di(C₁₋₆alkyl)amino-C₁₋₆alkyl, aryl-C₁₋₆alkyl or heteroaryl-C₁₋₆alkyl; R² is C₁₋₆alkyl; R³ is hydrogen or C₁₋₆alkyl; R¹⁰ is hydrogen or C₁₋₆alkyl; R¹¹ is hydrogen, C₁₋₆alkyl or di(C₁₋₆alkyl)aminoC₁₋₆alkyl; Ar is aryl or heteroaryl which aryl or heteroaryl is unsubstituted or substituted with one to three substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, cyano, trifluoromethyl, trifluoromethoxy, difluoromethoxy, fluoromethoxy and —N(R¹²)R¹³, wherein R¹² and R¹³ are the same or different, and independently are hydrogen or C₁₋₆alkyl. More preferable are the compound represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl or aryl-C₁₋₆alkyl; R² is C₁₋₆alkyl; R³ is hydrogen or C₁₋₆alkyl; R¹⁰ is hydrogen or C₁₋₆alkyl; R¹¹ is hydrogen or C₁₋₆alkyl; Ar is phenyl which phenyl is unsubstituted or substituted with one to three substituents, which are the same or different, selected from the group consisting of halogen, C₁₋₃alkyl, C₁₋₃alkoxy, C₁₋₃alkylthio, trifluoromethyl and —N(R¹²)R¹³, wherein R¹² and R¹³ are the same or different, and independently are hydrogen or C₁₋₃alkyl. More preferable are the compound represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl or aryl-C₁₋₆alkyl; R² is C₁₋₃alkyl; R³ is C₁₋₃alkyl; R¹⁰ is hydrogen; R¹¹ is hydrogen; Ar is phenyl which phenyl is substituted with 2 or 3 substituents, which are the same or different, selected from the group consisting of halogen or C₁₋₃alkyl.

The preferable bond between X and Y is a double bond.

The preferable R² is C₁₋₆alkyl. More preferable R² is methyl.

The preferable R³ is C₁₋₆alkyl. More preferable R³ is ethyl.

The preferable R¹⁰ is hydrogen.

The preferable R¹¹ is hydrogen.

The preferable Ar is phenyl which phenyl is substituted with one to three substituents, which are the same or different, selected from the group consisting of halogen, C₁₋₃alkyl, C₁₋₃alkoxy, C₁₋₃alkylthio, trifluoromethyl and —N(R¹²)R¹³, wherein R¹² and R¹³ are the same or different, and independently are hydrogen or C₁₋₃alkyl. The more preferable Ar is phenyl which phenyl is substituted with 2 or 3 substituents, which are the same or different, selected from the group consisting of halogen or C₁₋₃alkyl.

The compound of the formula [I] can be produced, for example, by the process shown in the following reaction schemes 1-3 (in the following reaction schemes, R¹, R², R³, R¹¹ and Ar are as defined above, L¹ and L² are the same or different, selected from the group consisting of chloro, bromo, iodo, methanesulfonyloxy, benzenesulfonyloxy, toluenesulfonyloxy or trifluoromethanesulfonyloxy group, L³ is chloro, bromo or iodo, R^{a} is C₁₋₆alkyl, R^{b} is C₁₋₆alkyl, R^{c} is C₁₋₆alkyl, C₃₋₆cycloalkyl, aryl or heteroaryl, R^{d} is hydrogen or C₁₋₅alkyl).

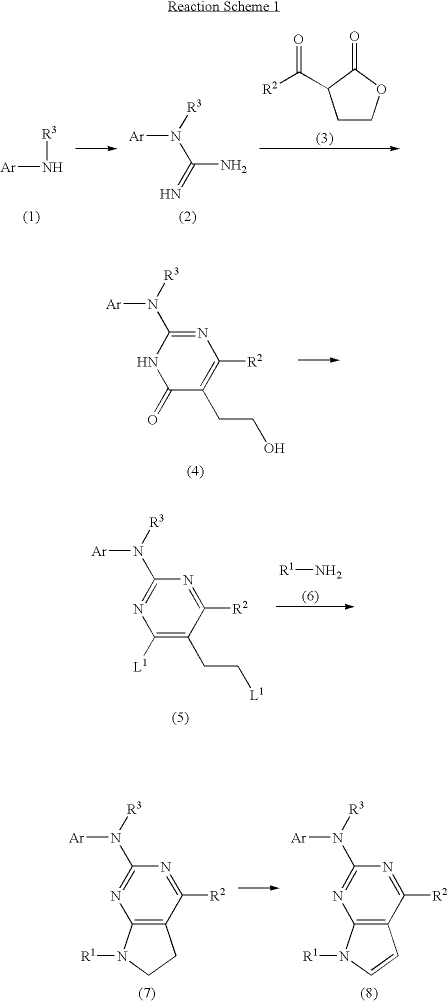

Compound (7) and (8), the compounds in the present invention, can be prepared by the method shown in reaction scheme 1. Compound (1) can be transformed to (2) by using a reagent for conversion of amine to guanidine in the presence or absence of a base in an inert solvent. Treatment of compound (2) with compound (3) can provide compound (4) in the presence or absence of a base in an inert solvent. Compound (4) can be converted to compound (5) using a halogenating reagent or a sulfonating reagent in the presence or absence of a base in an inert solvent or without using a solvent. Compound (5) can be treated with compound (6) to form compound (7) in the presence or absence of a base in an inert solvent. Treatment of compound (7) with an oxidizing agent in an inert solvent can give compound (8). When R³ in compound (7) [or (8)] is hydrogen, treatment of compound (7) [or (8)] with an alkylating reagent in the presence or absence of a base in an inert solvent can provide the N-alkylated compound (R³=C₁₋₆alkyl).

Herein, the reagent for conversion of amine to guanidine includes, for example, cyanamide, S-alkylthiouronium salt and its derivatives, aminoiminosulfonic acids, 3,5-dimethylpyrazole-1-carboxamidine nitrate, pyrazole-1-carboxamidine hydrochloride and the like. The base includes, for example, amines such as triethylamine, N,N-diisopropylethylamine, pyridine, N,N-dimethylaniline, N,N-diethylaniline and the like; inorganic bases such as sodium carbonate, potassium carbonate, sodium hydrogencarbonate, potassium hydrogencarbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide and the like; and Grignard reagents such as methyl magnesium bromide and the like. The halogenating reagent includes, for example, phosphoryl chloride, phosphoryl bromide, phosphorous pentachloride, phosphorous trichloride, phosphorous pentabromide, phosphorous tribromide, thionyl chloride, thionyl bromide, oxalyl chloride, oxalyl bromide and the like. The sulfonating reagent includes, for example, p-toluenesulfonyl chloride, methanesulfonyl chloride, p-toluenesulfonic anhydride, methansulfonic anhydride, trifluoromethanesulfonic anhydride, N-phenylbis(trifluoromethanesulfonimide) and the like. The oxidizing agent includes, for example, manganese dioxide, potassium permanganate, palladium and the like. The inert solvent includes, for example, alcohols such as methanol, ethanol, isopropyl alcohol, ethylene glycol and the like; ethers such as diethyl ether, diisopropyl ether, tetrahydrofuran, 1,4-dioxane, 1,2-dimethoxyethane and the like; hydrocarbons such as benzene, toluene and the like; esters such as ethyl acetate, ethyl formate and the like; ketones such as acetone, methylethylketone and the like; amides such as N,N-dimethylformamide, N-methylpyrrolidone, N,N-dimethylacetamide and the like; acetonitrile; dichloromethane; chloroform; dimethyl sulfoxide; pyridine; water; and mixtures of solvents selected from these inert solvents.

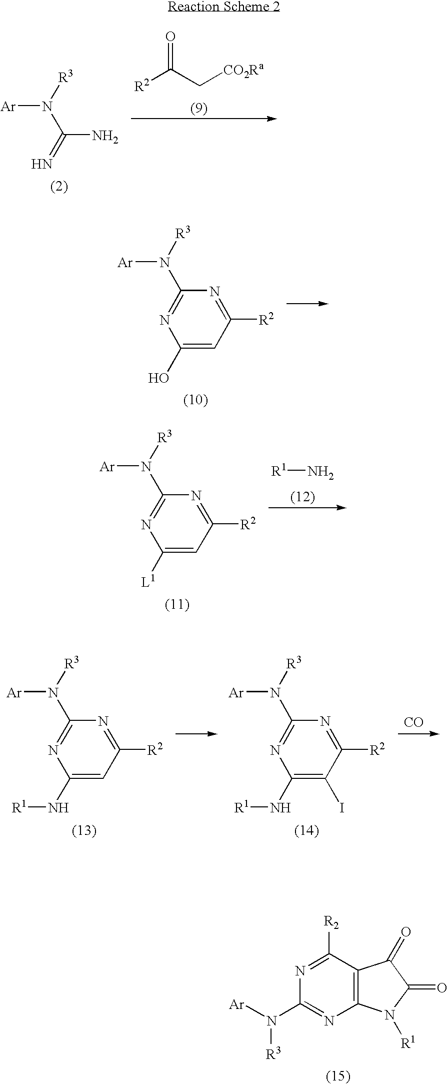

Compound (15), the compound in the present invention, can be prepared by the method shown in reaction scheme 2. Compound (2), synthesized in the same manner as shown in reaction scheme 1, can be converted to compound (10) by reacting with compound (9) in the presence or absence of a base in an inert solvent. Treatment of compound (10) with a halogenating reagent or a sulfonating reagent in the presence or absence of a base in an inert solvent or without using a solvent can provide compound (11). Compound (11) can be reacted with compound (12) in the presence or absence of a base in an inert solvent to form compound (13). Introduction of an iodine atom on the pyrimidine ring of compound (13) can be carried out in an inert solvent by using a conventional reagent for introducing an iodine atom such as iodine, iodine monochloride or the like. Compound (14) can be converted to compound (15) using a palladium catalyst, such as palladium (II) acetate, tetrakis(triphenylphosphine)palladium(0) or the like, under a carbon oxide atmosphere in the presence or absence of a base and a ligand in an inert solvent. Herein, the base includes, for example, amines such as triethylamine, N,N-diisopropylethylamine, pyridine, N,N-dimethylaniline, N,N-diethylaniline and the like; inorganic bases such as sodium carbonate, potassium carbonate, sodium hydrogencarbonate, potassium hydrogencarbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide and the like; and Grignard reagents such as methyl magnesium bromide and the like. The halogenating reagent includes, for example, phosphoryl chloride, phosphoryl bromide, phosphorous pentachloride, phosphorous trichloride, phosphorous pentabromide, phosphorous tribromide, thionyl chloride, thionyl bromide, oxalyl chloride, oxalyl bromide and the like. The sulfonating reagent includes, for example, p-toluenesulfonyl chloride, methanesulfonyl chloride, p-toluenesulfonic anhydride, methansulfonic anhydride, trifluoromethanesulfonic anhydride, N-phenylbis(trifluoromethanesulfonimide) and the like. The ligand includes, for example, triphenylphosphine, 1,3-bis(diphenylphosphono)propane and the like. The inert solvent includes, for example, alcohols such as methanol, ethanol, isopropyl alcohol, ethylene glycol and the like; ethers such as diethyl ether, diisopropyl ether, tetrahydrofuran, 1,4-dioxane, 1,2-dimethoxyethane and the like; hydrocarbons such as benzene, toluene and the like; esters such as ethyl acetate, ethyl formate and the like; ketones such as acetone, methylethylketone and the like; amides such as N,N-dimethylformamide, N-methylpyrrolidone, N,N-dimethylacetamide and the like; acetonitrile; dichloromethane; chloroform; dimethyl sulfoxide; pyridine; water; and mixtures of solvents selected from these inert solvents.

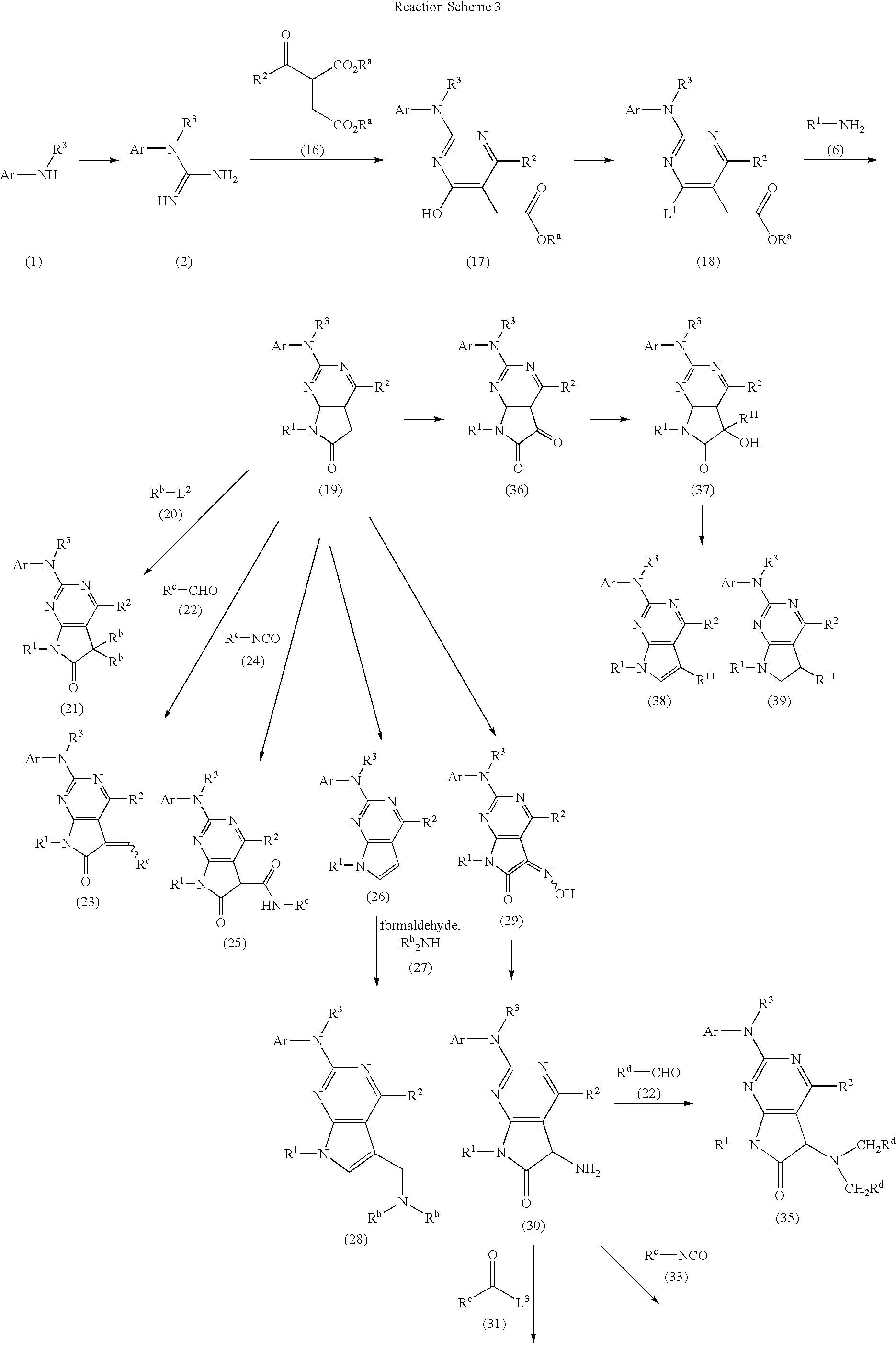

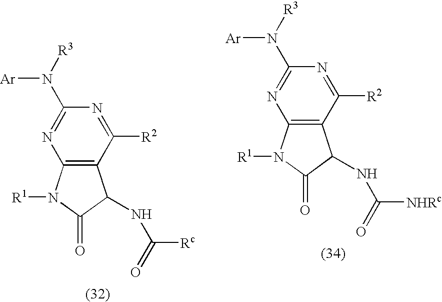

Compound (19), (21), (23), (25), (26), (28), (29), (30), (32), (34), (35), (36), (37), (38) and (39), the compounds in the present invention, can be prepared by the method shown in reaction scheme 3. Compound (2) can be prepared in the same manner as shown in reaction scheme 1. Compound (17) was given by reacting compound (2) with compound (16) in the presence or absence of a base in an inert solvent. Preparation of compound (17) from compound (1) may be performed in one pot continuously. Conversion of compound (17) to compound (18) can be carried out in the same method for the conversion of compound (4) to compound (5) in reaction scheme 1. Treatment of compound (18) with amine (6) in the presence or absence of a base in an inert solvent can provide compound (19). Compound (19) can be transformed to compound (21) by treatment with a base and an alkylating reagent (20) in an inert solvent. Reacting compound (19) with aldehyde (22) in the presence of a base in an inert solvent gave an alkylidene compound (23). Compound (25) can be provided by acylation of compound (19) with isocyanate (24) in the presence of base in an inert solvent. Reduction of a carbonyl group in compound (19) with a reducing agent in an inert solvent can provide compound (26). Compound (28) can be produced by Mannich reaction of compound (26) using an amine (27) and formaldehyde. Conversion of compound (19) to oxime (29) can be performed by reacting compound (19) with a nitrite derivative in the presence or absence of an acid in an inert solvent. Following reduction of the oxime group in compound (29) with a reducing agent in an inert solvent can give compound (30). Acylation of the amino group in compound (30) by using an acylating agent (31) in an inert solvent can give compound (32). Urea derivatives (34) can be produced by reacting compound (30) with an isocyanate (33) in an inert solvent. Reacting a mixture of compound (30) and an aldehyde (22) in the presence of a catalyst for hydrogenation under hydrogen atmosphere or in the presence of a reducing agent in an inert solvent can provide compound (35). Compound (36) can be provided by oxidation of compound (19) with an oxidizing agent in an inert solvent. Treatment of compound (36) with a Grignard reagent or alkyl lithium in an inert solvent can give compound (37). Reduction of compound (37) with a reducing agent in an inert solvent can provide compound (38) and/or compound (39).

Herein, the base includes, for example, amines such as triethylamine, N,N-diisopropylethylamine, pyridine 1,8-diazabicyclo[5.4.0]undec-7-ene and the like; inorganic bases such as sodium carbonate, potassium carbonate, sodium hydrogencarbonate, potassium hydrogen carbonate, sodium hydroxide, potassium hydroxide, barium hydroxide, sodium hydride and the like; metal alcoholates such as sodium methoxide, sodium ethoxide, potassium tert-butoxide and the like; metal amides such as sodium amide, lithium diisopropylamide, lithium hexamethyldisilazanide, sodium hexamethyldisilazanide, potassium hexamethyldisilazanide and the like. The acid includes, for example, includes inorganic acids such as sulfuric acid, hydrochloric acid, hydrobromic acid, phosphoric acid, nitric acid and the like; organic acids such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid, naphthalene-2-sulfonic acid and the like. The reducing agent includes, for example, lithium borohydride, sodium borohydride, calcium borohydride, lithium triethylborohydride, lithium tri-sec-butylborohydride, potassium tri-sec-butylborohydride, zinc borohydride, borane, lithium trimethoxyborohydride, lithium triacetoxyborohydride, tetramethylammonium borohydride, lithium aluminum hydride, sodium aluminum hydride, sodium bis(2-methoxyethoxy)aluminum hydride, diisobutylaluminum hydride, trichlorosilane and the like. The oxidizing agent includes, for example, manganese dioxide, potassium permanganate, palladium and the like. The catalyst for hydrogenation includes, for example, palladium, nickel and the like. The Grignard reagent includes, for example, methylmagnesium iodide, methylmagnesium bromide, methylmagnesium chloride, ethylmagnesium bromide, ethylmagnesium chloride. The alkyl lithium includes, for example, methyllithium, ethyllithium, butyllithium and the like. The nitrite derivative includes, for example, nitrite salts such as sodium nitrite, potassium nitrite and the like; organic nitrite derivatives such as butyl nitrite, isobutylnitrite, isoamylnitrite and the like. The inert solvent includes, for example, alcohols such as methanol, ethanol, isopropyl alcohol, ethylene glycol and the like; ethers such as diethyl ether, diisopropyl ether, tetrahydrofuran, 1,4-dioxane, 1,2-dimethoxyethane and the like; hydrocarbons such as benzene, toluene and the like; esters such as ethyl acetate, ethyl formate and the like; ketones such as acetone, methylethylketone and the like; amides such as N,N-dimethylformamide, N-methylpyrrolidone, N,N-dimethylacetamide and the like; acetonitrile; dichloromethane; chloroform; dimethyl sulfoxide; pyridine; water; and mixtures of solvents selected from these inert solvents.

The compound of the present invention can be converted to a salt with an acid in an inert solvent. The acid includes inorganic acids such as sulfuric acid, hydrochloric acid, hydrobromic acid, phosphoric acid, nitric acid and the like; organic acids such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid, naphthalene-2-sulfonic acid and the like. The inert solvent includes, for example, alcohols such as methanol, ethanol, isopropyl alcohol, ethylene glycol and the like; ethers such as diethyl ether, tetrahydrofuran, 1,4-dioxane, 1,2-dimethoxyethane and the like; hydrocarbons such as benzene, toluene and the like; amides such as N,N-dimethylformamide, N-methylpyrrolidone, N,N-dimethylacetamide and the like; esters such as ethyl acetate, ethyl formate and the like; ketones such as acetone, methylethylketone and the like; acetonitrile; dichloromethane; chloroform; dimethyl sulfoxide; pyridine; water; and mixtures of solvents selected from these inert solvents.

The compound of the present invention is useful as a therapeutic or prophylactic agent for diseases in which CRF is considered to be involved. For this purpose, the compound of the present invention can be formulated into tablets, pills, capsules, granules, powders, solutions, emulsions, suspensions, injections and the like by a conventional preparation technique by adding conventional fillers, binders, disintegrators, pH-adjusting agents, solvents, etc.

The compound of the present invention can be administered to an adult patient in a dose of 0.1 to 500 mg per day in one portion or several portions orally or parenterally. The dose can be properly increased or decreased depending on the kind of a disease and the age, body weight and symptom of a patient.

### PREFERRED EMBODIMENTS OF THE INVENTION

The present invention is concretely explained with reference to the following examples and a test example, but is not limited thereto.

### REFERENCE EXAMPLE 1

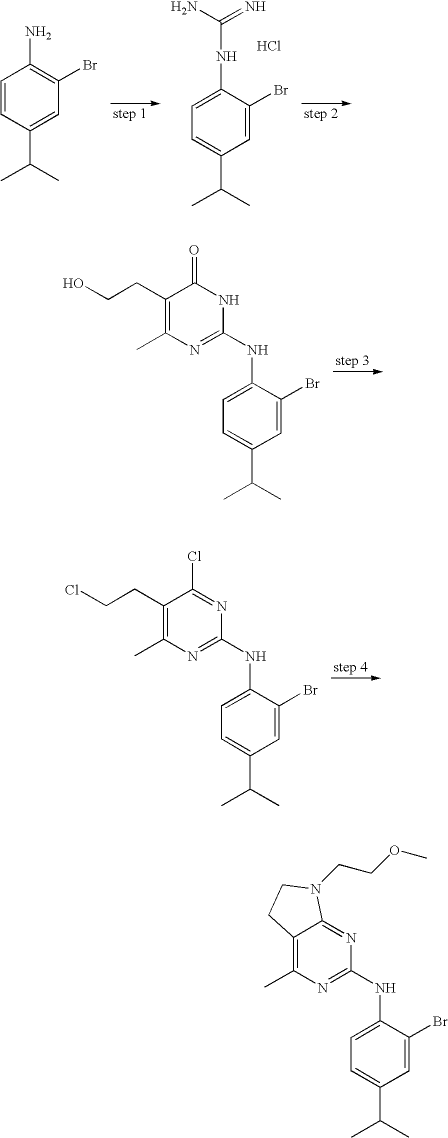

### Synthesis of (2-bromo-4-isopropyl-phenyl)-[7-(2-methoxy-ethyl)-4-methyl-6,7-dihydro-5H-pyrrolo[2,3-d]pyrimidine-2-yl]-amine

(Step 1) In a flask, equipped with a Dean Stark apparatus, a mixture of 2-bromo-4-isopropyl aniline (50 g) and cyanamide (39 g) in ethyl acetate (850 ml) and ethanol (110 ml) was stirred at room temperature. A solution of 1M HCl in ether was added and the reaction mixture was stirred for 1 h. The ether was distillated and the reaction mixture was stirred and refluxed overnight. The reaction mixture was cooled to room temperature and diluted with ether (1000 ml) to give a solid. The solid was filtered off, washed with acetonitrile and dried to give 40 g of N-(2-bromo-4-isopropyl-phenyl)-guanidine hydrochloride. The filtrate was concentrated under reduced pressure and the residue was crystallized from acetonitrile to provide a second fraction (8 g) of the product.

(Step 2) A mixture of N-(2-bromo-4-isopropyl-phenyl)-guanidine hydrochloride (48 g), 2-acetylbutyrolactone (30 g) and triethylamine (33 g) in ethanol (170 ml) was stirred and refluxed overnight. The solvent was evaporated and the residue purified by a silica gel column chromatography (eluent: dichloromethane/ammonia 7M in methanol=95:5) to give 2-(2-bromo-4-isopropyl-phenylamino)-5-(2-hydroxy-ethyl)-6-methyl-3H-pyrimidin-4-one (25 g) as a solid.

(Step 3) A mixture of 2-(2-bromo-4-isopropyl-phenylamino)-5-(2-hydroxy-ethyl)-6-methyl-3H-pyrimidin-4-one (23.5 g) and phosphorus oxychloride (300 ml) was stirred at 60° C. overnight. The reaction mixture was concentrated under reduced pressure, washed with water and extracted with dichloromethane. The organic layer was dried over magnesium sulfate, filtered and the solvent was evaporated. The residue was purified by a silica gel column chromatography (eluent: dichloromethane=100) to give (2-bromo-4-isopropyl-phenyl)-[4-chloro-5-(2-chloro-ethyl)-6-methyl-pyrimidin-2-yl]-amine (22 g) as a solid.

(Step 4) A mixture of (2-bromo-4-isopropyl-phenyl)-[4-chloro-5-(2-chloro-ethyl)-6-methyl-pyrimidin-2-yl]-amine (6 g) and 2-methoxyethylamine (1.5 g) in dioxane (50 ml) was stirred at 120° C. overnight. The solvent was evaporated and the residue was purified by a silica gel column chromatography (eluent: dichloromethane/methanol=97:3) to give (2-bromo-4-isopropyl-phenyl)-[7-(2-methoxy-ethyl)-4-methyl-6,7-dihydro-5H-pyrrolo[2,3-d]pyrimidine-2-yl]-amine (3.6 g).

### REFERENCE EXAMPLE 2

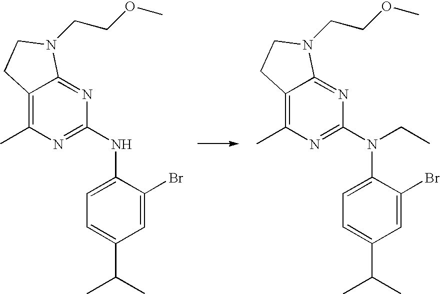

### Synthesis of (2-bromo-4-isopropyl-phenyl)-ethyl-[7-(2-methoxy-ethyl)-4-methyl-6,7-dihydro-5H-pyrrolo[2,3-d]pyrimidin-2-yl]-amine

A mixture of (2-bromo-4-isopropyl-phenyl)-[7-(2-methoxy-ethyl)-4-methyl-6,7-dihydro-5H-pyrrolo[2,3-d]pyrimidine-2-yl]-amine (0.6 g), iodoethane (0.3 g) and sodium hydride (0.3 g) in tetrahydrofuran (20 ml) was stirred at 60° C. for 4 h. Ethyl acetate (40 ml) and a solution of sodium hydroxide 0.5M (40 ml) were added. The organic layer was separated and the aqueous layer was extracted with ethyl acetate. The combined organic layers were washed with water, separated, dried over magnesium sulfate, filtered and the solvent was evaporated. The residue was purified by a silica gel column chromatography (eluent: dichloromethane/methanol=97:3) to give (2-bromo-4-isopropyl-phenyl)-ethyl-[7-(2-methoxy-ethyl)-4-methyl-6,7-dihydro-5H-pyrrolo[2,3-d]pyrimidin-2-yl]-amine (0.46 g).

### EXAMPLE 1

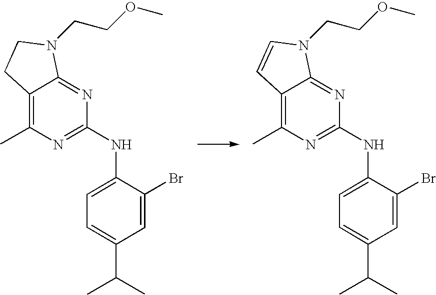

### Synthesis of (2-bromo-4-isopropyl-phenyl)-[7-(2-methoxy-ethyl)-4-methyl-7H-pyrrolo[2,3-d]pyrimidin-2-yl]-amine (1-010)

A mixture of (2-bromo-4-isopropyl-phenyl)-[7-(2-methoxy-ethyl)-4-methyl-6,7-dihydro-5H-pyrrolo[2,3-d]pyrimidine-2-yl]-amine (1.7 g) and manganese(IV) oxide (1.5 g) in dioxane (25 ml) was stirred and refluxed for 4 h. The reaction mixture was cooled and filtered over decalite. The filtrate was concentrated under reduced pressure and purified by a silica gel column chromatography (eluent: dichloromethane/methanol=99:1) to give (2-bromo-4-isopropyl-phenyl)-[7-(2-methoxy-ethyl)-4-methyl-7H-pyrrolo[2,3-d]pyrimidin-2-yl]-amine (0.31 g).

### EXAMPLE 2

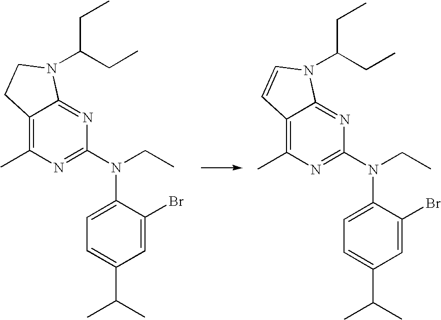

### Synthesis of (2-bromo-4-isopropyl-phenyl)-ethyl-[7-(1-ethyl-propyl)-4-methyl-7H-pyrrolo[2,3-d]pyrimidin-2-yl]-amine (1-003)

A mixture of (2-bromo-4-isopropyl-phenyl)-ethyl-[7-(1-ethyl-propyl)-4-methyl-6,7-dihydro-5H-pyrrolo[2,3-d]pyrimidin-2-yl]-amine (0.4 g) and manganese(IV) oxide (0.4 g) in dioxane (10 ml) was stirred and refluxed for 3 h. The reaction mixture was cooled and filtered over decalite. The filtrate was concentrated under reduced pressure and purified by a silica gel column chromatography (eluent: dichloromethane/methanol=99:1) to give (2-bromo-4-isopropyl-phenyl)-ethyl-[7-(1-ethyl-propyl)-4-methyl-7H-pyrrolo[2,3-d]pyrimidin-2-yl]-amine (0.37 g).

### EXAMPLE 3

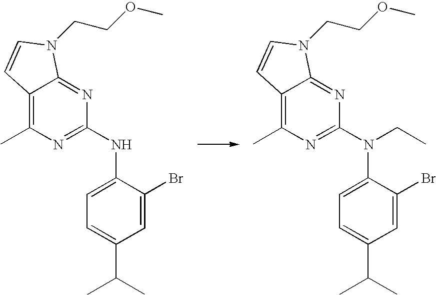

### Synthesis of (2-bromo-4-isopropyl-phenyl)-ethyl-[7-(2-methoxy-ethyl)-4-methyl-7H-pyrrolo[2,3-d]pyrimidin-2-yl]-amine (1-002)

A mixture of (2-bromo-4-isopropyl-phenyl)-[7-(2-methoxy-ethyl)-4-methyl-7H-pyrrolo[2,3-d]pyrimidin-2-yl]-amine (0.9 g), iodoethane (0.4 g) and sodium hydride (0.4 g) in tetrahydrofuran (20 ml) was stirred at 60° C. for 4 h. Ethyl acetate (50 ml) and a solution of sodium hydroxide 0.5M (50 ml) were added. The organic layer was separated and the aqueous layer was extracted with ethyl acetate. The combined organic layers were washed with water, separated, dried over magnesium sulfate, filtered and the solvent was evaporated. The residue was purified by a silica gel column chromatography (eluent: dichloromethane/methanol=98:2) to give (2-bromo-4-isopropyl-phenyl)-ethyl-[7-(2-methoxy-ethyl)-4-methyl-7H-pyrrolo[2,3-d]pyrimidin-2-yl]-amine (0.32 g).

### EXAMPLE 4

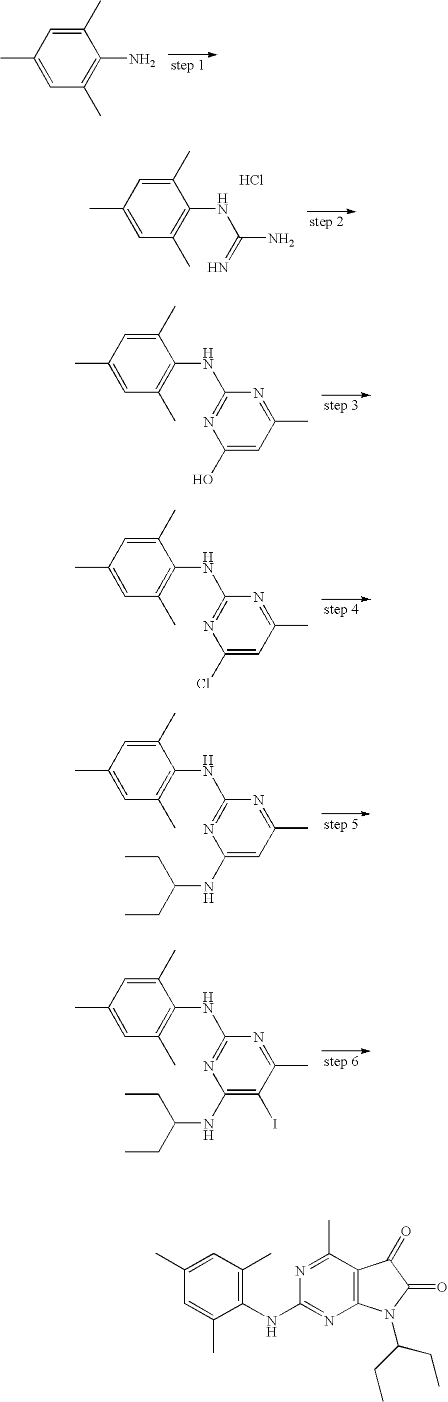

### Synthesis of 7-(1-ethyl-propyl)-4-methyl-2-(2,4,6-trimethyl-phenylamino)-7H-pyrrolo[2,3-d]pyrimidine-5,6-dione (4-002)

(Step 1) is analogous to (Reference example 1, step 1).

(Step 2) A mixture of N-(2,4,6-trimethyl-phenyl)-guanidine hydrochloride (14.8 g), ethyl acetoacetate (39 g) and potassium carbonate (14 g) in ethanol (300 ml) was stirred and refluxed for 16 h. The solvent was evaporated and the residue purified by a silica gel column chromatography (eluent: dichloromethane/methanol=98:2). The product was crystallized from hexane, filtered and dried to provide 6-methyl-2-(2,4,6-trimethyl-phenylamino)-pyrimidine-4-ol (15 g).

(Step 3) A mixture of 6-methyl-2-(2,4,6-trimethyl-phenylamino)-pyrimidine-4-ol (15 g) and phosphorus oxychloride (200 ml) was stirred and refluxed for 16 h. The reaction mixture was concentrated under reduced pressure and the residue was dissolved in dichloromethane. Water was added and the mixture was alkalified with potassium carbonate. The organic layer was washed with water, dried over magnesium sulfate, filtered and evaporated. The residue was purified by a silica gel column chromatography (eluent: dichloromethane=100) to give (4-chloro-6-methyl-pyrimidine-2-yl)-(2,4,6-trimethyl-phenyl)-amine (11 g).

(Step 4) A mixture of (4-chloro-6-methyl-pyrimidine-2-yl)-(2,4,6-trimethyl-phenyl)-amine (7.5 g), 3-ethyl-propylamine (3.5 g) and potassium carbonate (3.5 g) in acetonitrile was stirred at 125° C. for 2 days. The solvent was evaporated and the residue was dissolved in water and extracted with dichloromethane. The organic layer was dried over magnesium sulfate and filtered. The filtrate was concentrated under reduced pressure and purified by a silica gel column chromatography (eluent: dichloromethane/7M ammonia in methanol=98:2). The product was crystallized from isopropyl ether, filtered and dried to give N⁴-(1-ethyl-propyl)-6-methyl-N²-(2,4,6-trimethyl-phenyl)-pyrimidine-2,4-diamine (3.1 g).

(Step 5) To a solution of N⁴-(1-ethyl-propyl)-6-methyl-N²-(2,4,6-trhnethyl-phenyl)-pyrimidine-2,4-diamine (3.1 g) in methanol (30 ml) at room temperature was added dropwise a 1M solution of iodine monochloride in dichloromethane (10 ml). The reaction mixture was stirred for 1 h and concentrated under reduced pressure. The residue was purified by silica gel column chromatography (eluent: dichloromethane/methanol=98:2), crystallized from isopropyl ether, filtered and dried to provide N⁴-(1-ethyl-propyl)-5-iodo-6-methyl-N²-(2,4,6-trimethyl-phenyl)-pyrimidine-2,4-diamine (2.6 g).

(Step 6) A mixture of N⁴-(1-ethyl-propyl)-5-iodo-6-methyl-N²-(2,4,6-trimethyl-phenyl)-pyrimidine-2,4-diamine (0.5 g), palladium(II) acetate (0.02 g), 1,3-bis(diphenylphosphino)propane (0.08 g) and triethylamine (1 g) in tetrahydrofuran (50 ml) was stirred under 60 atmosphere CO pressure, at 75° C. for 16 h. The solvent was evaporated and the residue was purified by a silica gel column chromatography (eluent: dichloromethane/methanol=95:5) to give 7-(1-ethyl-propyl)-4-methyl-2-(2,4,6-trimethyl-phenylamino)-7H-pyrrolo[2,3-d]pyrimidine-5,6-dione (0.12 g).

### EXAMPLE 5

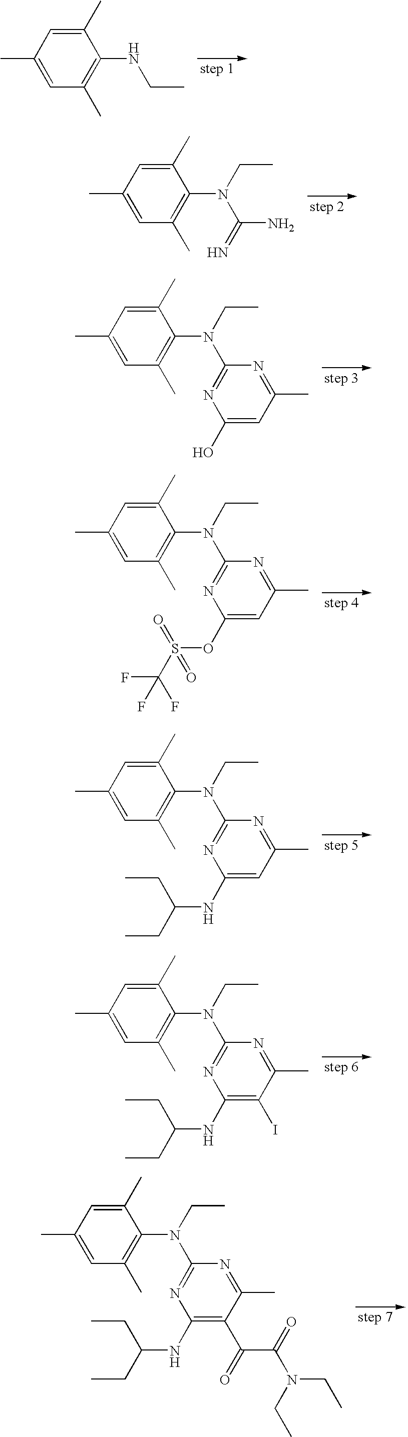

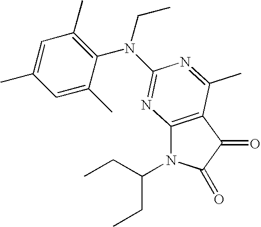

### Synthesis of 7-(1-ethyl-propyl)-2-[ethyl-(2,4,6-trimethyl-phenyl)-amino]-4-methyl-7H-pyrrolo[2,3-d]pyrimidine-5,6-dione (4-001)

(Step 1 and step 2) A mixture of ethyl-(2,4,6-trimethyl-phenyl)-amine (50 g) and cyanamide (21 g) in N-methylpyrrolidone (50 ml) was stirred at 150° C. for 1 h. The reaction mixture was cooled to room temperature. Ethanol (500 ml), ethyl acetoacetate (65 g) and potassium carbonate (37 g) were added and the mixture was stirred and refluxed for 16 h. The solvent was evaporated and the residue was dissolved in water and extracted with ethyl acetate (2×). The combined organic layers were washed with water, dried over magnesium sulfate and concentrated under reduced pressure. The residue was crystallized from isopropyl ether, filtered and dried to provide 2-[ethyl-(2,4,6-trimethyl-phenyl)-amino]-6-methyl-pyrimidin-4-ol (29 g). The filtrate was concentrated under reduced pressure and purified by a reversed phase column chromatography (eluent: ammonium acetate/acetonitrile) to give a second fraction of the product (7.7 g).

(Step 3) A mixture of 2-[ethyl-(2,4,6-trimethyl-phenyl)-amino]-6-methyl-pyrimidin-4-ol (2.7 g) and N,N-diisopropylethylamine (1.6 g) in dichloromethane (100 ml) was stirred under nitrogen at 0° C. Triflic anhydride (3.4 g) was added dropwise. The reaction mixture was brought to room temperature and stirred for 1 h. Water was added and the organic layer was dried over magnesium sulfate, filtered and evaporated to give trifluoro-methanesulfonic acid 2-[ethyl-(2,4,6-trimethyl-phenyl)-amino]-6-methyl-pyrimidin-4-yl ester (4.1 g).

(Step 4) is analogous to (example 4, step 4).

(Step 5) is analogous to (example 4, step 5).

(Step 6) A mixture of N²-ethyl-N⁴-(1-ethyl-propyl)-5-iodo-6-methyl-N²-(2,4,6-trimethyl-phenyl)-pyrimidine-2,4-diamine (0.5 g), palladium(II) acetate (0.02 g), 1,3-bis(diphenylphosphino)propane (0.08 g) and diethylamine (25 ml) in tetrahydrofuran (50 ml) was stirred under 60 atmosphere CO pressure, at 75° C. for 16 h. The solvent was evaporated and the residue was purified by a silica gel column chromatography (eluent: dichloromethane/methanol=95:5) to give N,N-diethyl-2-{4-(1-ethyl-propylamino)-2-[ethyl-(2,4,6-trimethyl-phenyl)-amino]-6-methyl-pyrimidin-5-yl}-2-oxo-acetamide (0.2 g).

(Step 7) N,N-diethyl-2-{4-(1-ethyl-propylamino)-2-[ethyl-(2,4,6-trimethyl-phenyl)-amino]-6-methyl-pyrimidin-5-yl}-2-oxo-acetamide (0.05 g) and a solution of 6M hydrochloric acid in 2-propanol (1 ml) were stirred at 150° C. for 30 minutes. The product was purified by a reversed phase column chromatography (eluent: ammonium acetate/acetonitrile) to give 7-(1-ethyl-propyl)-2-[ethyl-(2,4,6-trimethyl-phenyl)-amino]-4-methyl-7H-pyrrolo[2,3-d]pyrimidine-5,6-dione (0.006 g).

### EXAMPLE 6

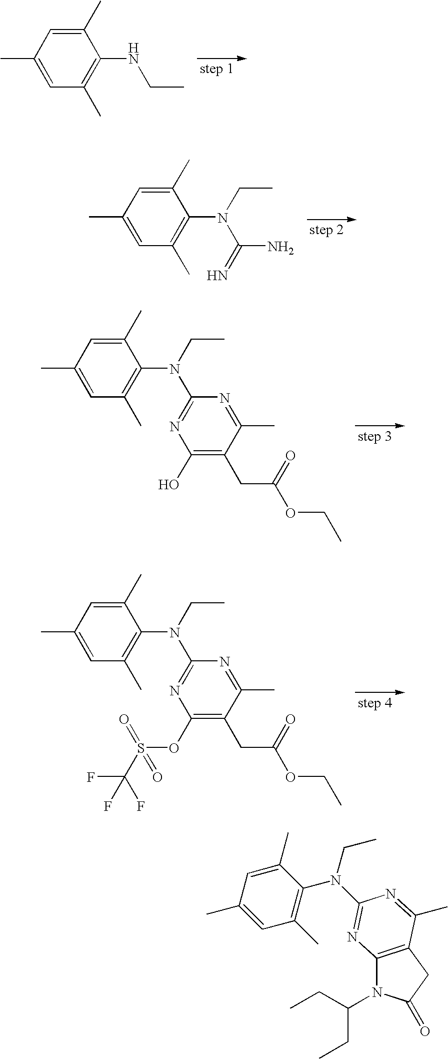

### Synthesis of 7-(1-ethyl-propyl)-2-[ethyl-(2,4,6-trimethyl-phenyl)-amino]-4-methyl-5,7dihydro-pyrrolo[2,3-d]pyrimidin-6-one (3-001)

(Step 1 and step 2) A mixture of ethyl-(2,4,6-trimethyl-phenyl)-amine (50 g) and cyanamide (21 g) in N-methylpyrrolidone (50 ml) was stirred at 150° C. for 1 h. The reaction mixture was cooled to room temperature. Ethanol (1000 ml), diethyl acetylsuccinate (65 g) and potassium carbonate (74 g) were added and the mixture was stirred and refluxed for 16 h. Diethyl acetylsuccinate (65 g) was added a second time and the reaction mixture was stirred and refluxed for 24 h. A solution of 6M hydrochloric acid in 2-propanol was added and the mixture was stirred at 60° C. for 24 h. The solvent was evaporated and water was added. The mixture was alkalified with a solution of potassium carbonate and extracted with ethyl acetate. The organic layer was dried over magnesium sulfate, filtered and concentrated under reduced pressure. The residue was purified by a silica gel column chromatography (eluent: dichloromethane/methanol=95:5) to provide {2-[ethyl-(2,4,6-trimethyl-phenyl)-amino]-4-hydroxy-6-methyl-pyrimidin-5-yl}-acetic acid ethyl ester (78 g).

(Step 3) is analogous to (example 5, step 3)

(Step 4) A mixture of {2-[ethyl-(2,4,6-trimethyl-phenyl)-amino]-4-methyl-6-trifluoromethanesulfonyloxy-pyrimidin-5-yl}-acetic acid ethyl ester (10 g), 1-ethyl-propylamine (4 g) and potassium carbonate (4 g) in acetonitrile (100 ml) was stirred at 125° C. for 72 h. The solvent was evaporated and the residue was dissolved in water and extracted with dichloromethane. The organic layer was dried over magnesium sulfate and evaporated to give 7-(1-ethyl-propyl)-2-[ethyl-(2,4,6-trimethyl-phenyl)-amino]-4-methyl-5,7dihydro-pyrrolo[2,3-d]pyrimidin-6-one (8 g).

### EXAMPLE 7

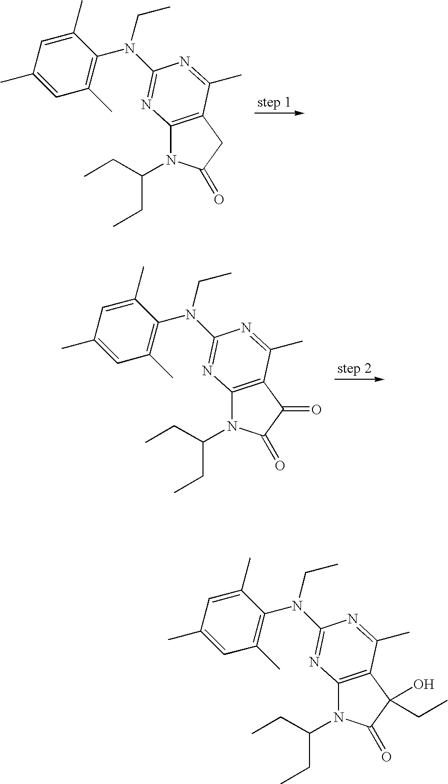

### Synthesis of 5-ethyl-7-(1-ethyl-propyl)-2-[ethyl-(2,4,6-trimethyl-phenyl)-amino]-5-hydroxy-4-methyl-5,7-dihydro-pyrrolo[2,3-d]pyrimidin-6-one (3-020)

(Step 1) A mixture of 7-(1-ethyl-propyl)-2-[ethyl-(2,4,6-trimethyl-phenyl)-amino]-4-methyl-5,7dihydro-pyrrolo[2,3-d]pyrimidin-6-one (0.6 g) and manganese(IV) oxide (0.5 g) in dichloromethane (2 ml) was stirred at room temperature for 16 h. The reaction mixture was filtered over decalite and the filtrate was concentrated under reduced pressure to give 7-(1-ethyl-propyl)-2-[ethyl-(2,4,6-trimethyl-phenyl)-amino]-4-methyl-7H-pyrrolo[2,3-d]pyrimidine-5,6-dione (0.1 g).

(Step 2) A solution of 7-(1-ethyl-propyl)-2-[ethyl-(2,4,6-trimethyl-phenyl)-amino]-4-methyl-7H-pyrrolo[2,3-d]pyrimidine-5,6-dione (0.15 g) in tetrahydrofuran (1.5 ml) under nitrogen was stirred at −20° C. 1 M ethylmagnesium bromide in tetrahydrofuran (0.5 ml) was added. The reaction mixture was brought to room temperature and stirred for 1 h. A solution of ammonium chloride (1 ml) was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 5-ethyl-7-(1-ethyl-propyl)-2-[ethyl-(2,4,6-trimethyl-phenyl)-amino]-5-hydroxy-4-methyl-5,7-dihydro-pyrrolo[2,3-d]pyrimidin-6-one (0.034 g).

### EXAMPLE 8

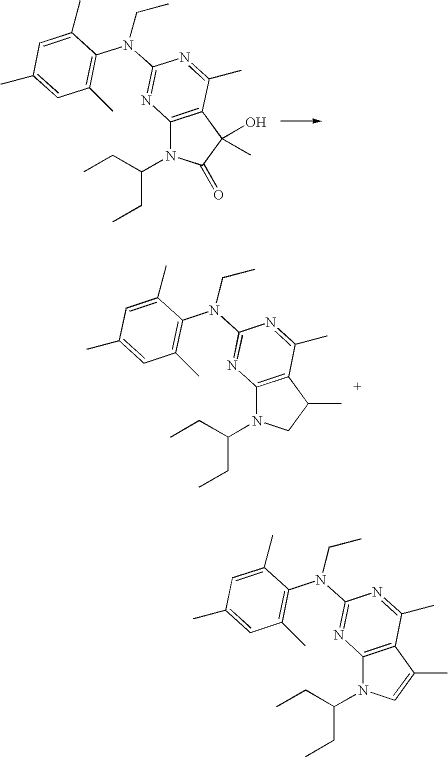

### Synthesis of ethyl-[7-(1-ethyl-propyl)-4,5-dimethyl-6,7-dihydro-5H-pyrrolo[2,3-d]pyrimidin-2-yl]-(2,4,6-trimethyl-phenyl)-amine (2-001) and ethyl-[7-(1-ethyl-propyl)-4,5-dimethyl-7H-pyrrolo[2,3-d]pyrimidin-2-yl]-(2,4,6-trimethyl-phenyl)-amine (1-015)

7-(1-ethyl-propyl)-2-[ethyl-(2,4,6-trimethyl-phenyl)-amino]-5-hydroxy-4,5-dimethyl-5,7-dihydro-pyrrolo[2,3-d]pyrimidin-6-one (0.8 g), prepared in the similar method as example 7, in tetrahydrofuran (20 ml) was stirred at 0° C. under nitrogen. Borane-tetrahydrofuran complex, 1M solution in tetrahydrofuran (14 ml) was added and the reaction mixture was stirred for 16 h. The solvent was evaporated, water and potassium carbonate were added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give ethyl-[7-(1-ethyl-propyl)-4,5-dimethyl-6,7-dihydro-5H-pyrrolo[2,3-d]pyrimidin-2-yl]-(2,4,6-trimethyl-phenyl)-amine (0.035 g) and ethyl-[7-(1-ethyl-propyl)-4,5-dimethyl-7H-pyrrolo[2,3-d]pyrimidin-2-yl]-(2,4,6-trimethyl-phenyl)-amine (0.011 g).

### EXAMPLE 9

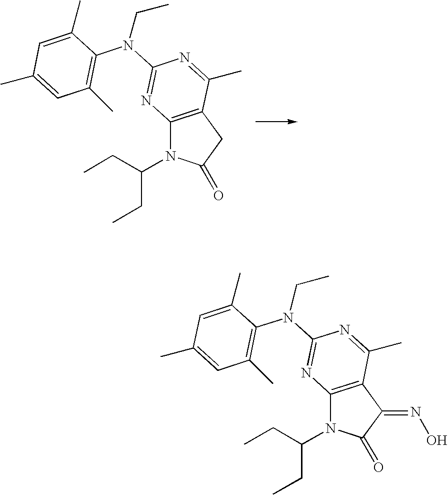

### Synthesis of 7-(1-ethyl-propyl)-2-[ethyl-(2,4,6-trimethyl-phenyl)-amino]-4-methyl-7H-pyrrolo[2,3-d]pyrimidine-5,6-dione 5-oxime (6-001)

A solution of 7-(1-ethyl-propyl)-2-[ethyl-(2,4,6-trimethyl-phenyl)-amino]-4-methyl-5,7-dihydro-pyrrolo[2,3-d]pyrimidin-6-one (1.3 g) in acetic acid (20 ml) was stirred at room temperature. Sodium nitrite (0.5 g) was added and 3 drops of water were added. The reaction mixture was stirred for 1 h, poured out into water and extracted with dichloromethane. The organic layer was dried over magnesium sulfate, filtered and evaporated to provide 7-(1-ethyl-propyl)-2-[ethyl-(2,4,6-trimethyl-phenyl)-amino]-4-methyl-7H-pyrrolo[2,3-d]pyrimidine-5,6-dione 5-oxime (1.4 g) as a mixture of the geometric isomers.

### EXAMPLE 10

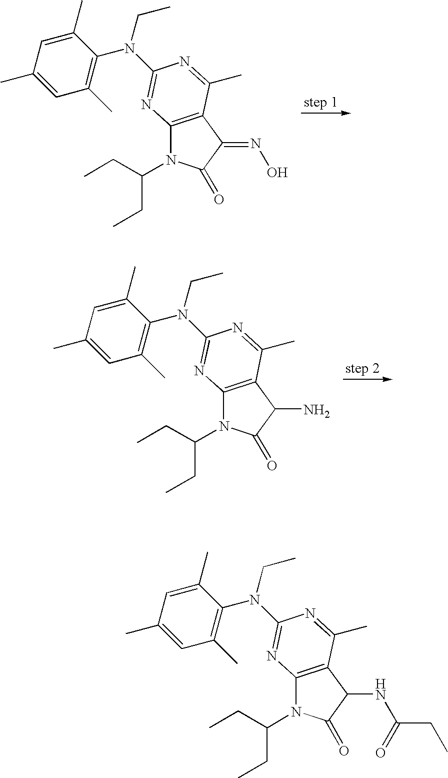

### Synthesis of N-{7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-yl}-propionamide (3-005)

(Step 1) 7-(1-ethyl-propyl)-2-[ethyl-(2,4,6-trimethyl-phenyl)-amino]-4-methyl-7H-pyrrolo[2,3-d]pyrimidine-5,6-dione 5-oxime (0.5 g) was hydrogenated with Raney Nickel in tetrahydrofuran (50 ml). The reaction mixture was filtered over decalite and the filtrate was concentrated under reduced pressure to give 5-amino-7-(1-ethyl-propyl)-2-[ethyl-(2,4,6-trimethyl-phenyl)-amino]-4-methyl-5,7-dihydro-pyrrolo[2,3-d]pyrimidin-6-one (0.5 g).

(Step 2) A mixture of 5-amino-7-(1-ethyl-propyl)-2-[ethyl-(2,4,6-trimethyl-phenyl)-amino]-4-methyl-5,7-dihydro-pyrrolo[2,3-d]pyrimidin-6-one (0.15 g), propionyl chloride (0.055 g) and triethylamine (0.1 g) in dichloromethane (2 ml) was stirred at room temperature for 16 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give N-{7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-yl}-propionamide (0.034 g).

### EXAMPLE 11

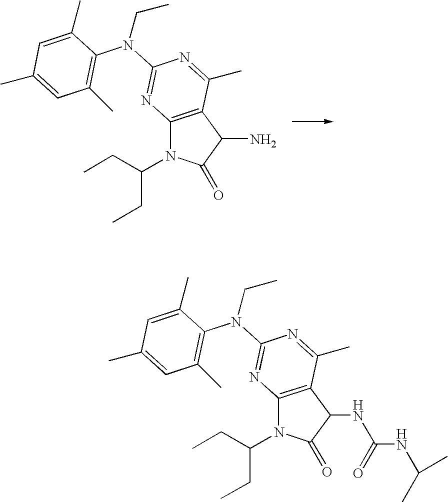

### Synthesis of 1-{7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-yl}-3-isopropyl-urea (3-007)

A mixture of 5-amino-7-(1-ethyl-propyl)-2-[ethyl-(2,4,6-trimethyl-phenyl)-amino]-4-methyl-5,7-dihydro-pyrrolo[2,3-d]pyrimidin-6-one (0.15 g), 2-isocyanato-propane (0.042 g), dimethylaminopropylamine (cat.) in dioxane (3 ml) was stirred at room temperature for 16 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 1-{7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-yl}-3-isopropyl-urea (0.015 g).

### EXAMPLE 12

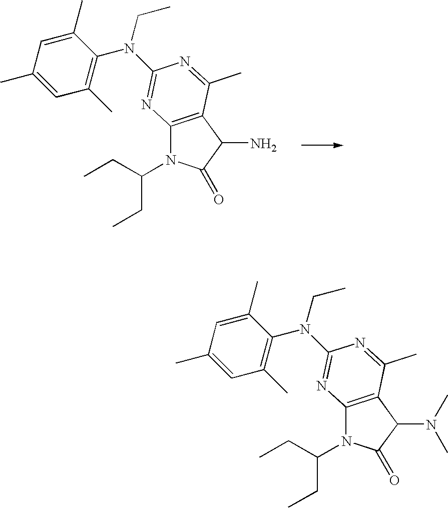

### Synthesis of 5-dimethylamino-7-(1-ethyl-propyl)-2-[ethyl-(2,4,6-trimethyl-phenyl)-amino]-4-methyl-5,7-dihydro-pyrrolo[2,3-d]pyrimidin-6-one (3-010)

A mixture of 5-amino-7-(1-ethyl-propyl)-2-[ethyl-(2,4,6-trimethyl-phenyl)-amino]-4-methyl-5,7-dihydro-pyrrolo[2,3-d]pyrimidin-6-one (0.1 g), paraformaldehyde (0.1 g), palladium on activated carbon, 10% (0.1 g) and thiophene 4% in diisopropylether (0.1 ml) in methanol (40 ml) was hydrogenated at 50° C. The reaction mixture was filtered over decalite and the filtrate was concentrated under reduced pressure. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 5-dimethylamino-7-(1-ethyl-propyl)-2-[ethyl-(2,4,6-trimethyl-phenyl)-amino]-4-methyl-5,7-dihydro-pyrrolo[2,3-d]pyrimidin-6-one (0.013 g).

### EXAMPLE 13

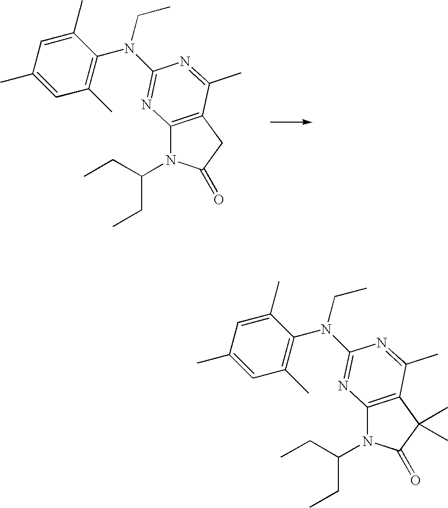

### Synthesis of 7-(1-ethyl-propyl)-2-[ethyl-(2,4,6-trimethyl-phenyl)-amino]-4,5,5-trimethyl-5,7dihydro-pyrrolo[2,3-d]pyrimidin-6-one (3-009)

A mixture of 7-(1-ethyl-propyl)-2-[ethyl-(2,4,6-trimethyl-phenyl)-amino]-4-methyl-5,7dihydro-pyrrolo[2,3-d]pyrimidin-6-one (0.15 g) and sodium hydride 50% (0.04 g) in tetrahydrofuran was stirred at room temperature for 15 minutes. Iodomethane (0.12 g) was added and the reaction mixture was stirred for 1 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 7-(1-ethyl-propyl)-2-[ethyl-(2,4,6-trimethyl-phenyl)-amino]-4,5,5-trimethyl-5,7dihydro-pyrrolo[2,3-d]pyrimidin-6-one (0.004 g).

### EXAMPLE 14

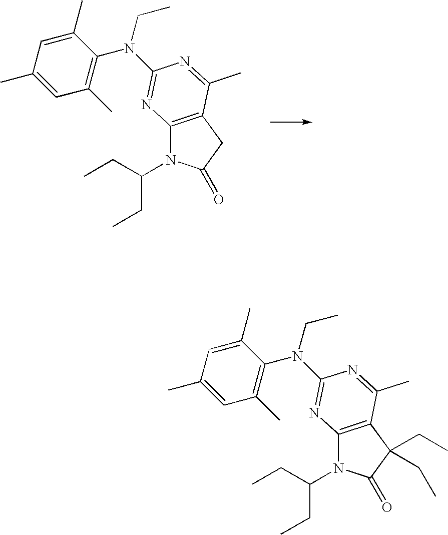

### Synthesis of 5,5-diethyl-7-(1-ethyl-propyl)-2-[ethyl-(2,4,6-trimethyl-phenyl)-amino]-4-methyl-5,7-dihydro-pyrrolo[2,3-d]pyrimidin-6-one (3-018)

A mixture of 7-(1-ethyl-propyl)-2-[ethyl-(2,4,6-trimethyl-phenyl)-amino]-4-methyl-5,7-dihydro-pyrrolo[2,3-d]pyrimidin-6-one (0.015 g) and sodium bis(trimethylsilyl)amide in dioxane (2 ml) was stirred at room temperature for 15 minutes under nitrogen. Bromoethane (0.087 g) was added and the reaction mixture was stirred at 60° C. for 1 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 5,5-diethyl-7-(1-ethyl-propyl)-2-[ethyl-(2,4,6-trimethyl-phenyl)-amino]-4-methyl-5,7-dihydro-pyrrolo[2,3-d]pyrimidin-6-one (0.018 g).

### EXAMPLE 15

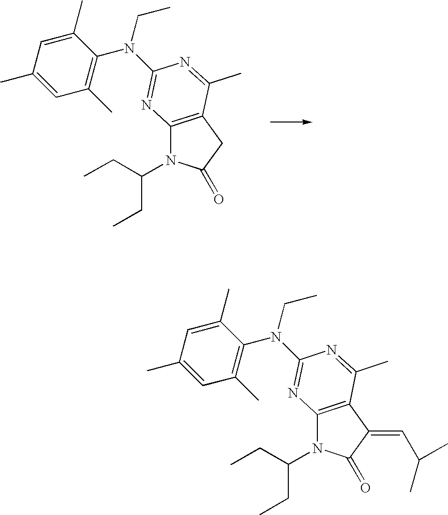

### Synthesis of 7-(1-ethyl-propyl)-2-[ethyl-(2,4,6-trimethyl-phenyl)-amino]-5-isobutylidene-4-methyl-5,7-dihydro-pyrrolo[2,3-d]pyrimidin-6-one (5-001)

A mixture of 7-(1-ethyl-propyl)-2-[ethyl-(2,4,6-trimethyl-phenyl)-amino]-4-methyl-5,7-dihydro-pyrrolo[2,3-d]pyrimidin-6-one (0.15 g), isobutyraldehyde (0.057 g) and piperidine in dioxane (1.5 ml) was stirred at 65° C. for 16 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 7-(1-ethyl-propyl)-2-[ethyl-(2,4,6-trimethyl-phenyl)-amino]-5-isobutylidene-4-methyl-5,7-dihydro-pyrrolo[2,3-d]pyrimidin-6-one (0.071 g) as a mixture of the geometric isomers.

### EXAMPLE 16

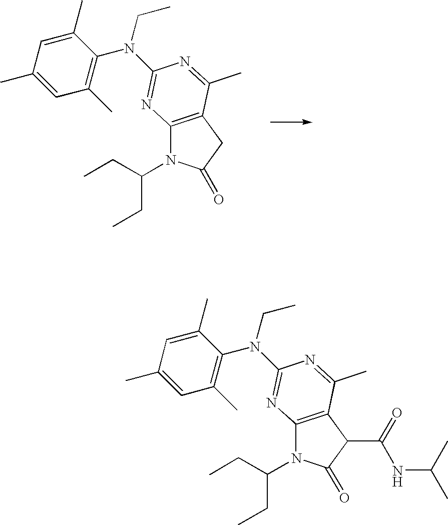

### Synthesis of 7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-carboxylic acid isopropylamide (3-022)

A mixture of 7-(1-ethyl-propyl)-2-[ethyl-(2,4,6-trimethyl-phenyl)-amino]-4-methyl-5,7dihydro-pyrrolo[2,3-d]pyrimidin-6-one (0.15 g), 2-isocyanato propane (0.042 g) and sodium bis(trimethylsilyl)amide in dioxane (2 ml) was stirred at 85° C. for 16 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give 7-(1-ethyl-propyl)-2-[ethyl-(2,4,6-trimethyl-phenyl)-amino]-4-methyl-6-oxo-6,7-dihydro-5H-pyrrolo[2,3-d]pyrimidin-5-carboxylic acid isopropylamide (0.114 g).

### EXAMPLE 17

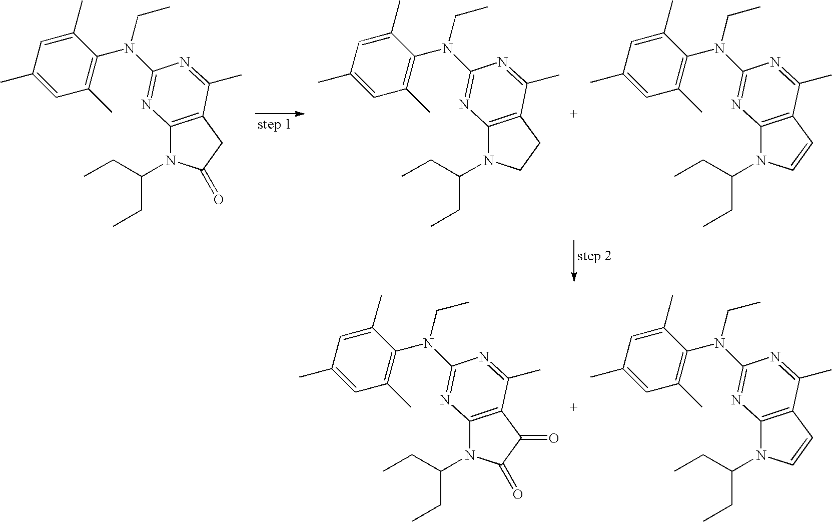

### Synthesis of ethyl-[7-(1-ethyl-propyl)-4-methyl-7H-pyrrolo[2,3-d]pyrimidin-2-yl]-(2,4,6-trimethyl-phenyl)-amine (1-008)

(Step 1) A solution of 7-(1-ethyl-propyl)-2-[ethyl-(2,4,6-trimethyl-phenyl)-amino]-4-methyl-5,7-dihydro-pyrrolo[2,3-d]pyrimidin-6-one (1 g) in tetrahydrofuran (20 ml) was stirred at 0° C. under nitrogen. Borane-tetrahydrofuran complex, 1M solution in tetrahydrofuran (12.5 ml) was added dropwise and the reaction mixture was stirred for 2 h at room temperature. Methanol/acetic acid 1:1 was added and the solvent was evaporated. The residue was dissolved in water, alkalified with potassium carbonate and extracted with dichloromethane. The organic layer was dried over magnesium sulfate, filtered and concentrated under reduced pressure to provide a mixture of ethyl-[7-(1-ethyl-propyl)-4-methyl-6,7-dihydro-5H-pyrrolo[2,3-d]pyrimidin-2-yl]-(2,4,6-trimethyl-phenyl)-amine (60%) and ethyl-[7-(1-ethyl-propyl)-4-methyl-7H-pyrrolo[2,3-d]pyrimidin-2-yl]-(2,4,6-trimethyl-phenyl)-amine (32%) (1 g). The residue was used without further purification.

(Step 2) A mixture of ethyl-[7-(1-ethyl-propyl)-4-methyl-6,7-dihydro-5H-pyrrolo[2,3-d]pyrimidin-2-yl]-(2,4,6-trimethyl-phenyl)-amine (60%) and ethyl-[7-(1-ethyl-propyl)-4-methyl-7H-pyrrolo[2,3-d]pyrimidin-2-yl]-(2,4,6-trimethyl-phenyl)-amine (32%) (1 g) and manganese(IV) oxide (5 g) in dichloromethane were stirred at room temperature for 76 h. The reaction mixture was filtered over decalite and the filtrate was concentrated under reduced pressure. The residue was purified by a silica gel column chromatography (eluent: dichloromethane/methanol=98:2) to give ethyl-[7-(1-ethyl-propyl)-4-methyl-7H-pyrrolo[2,3-d]pyrimidin-2-yl]-(2,4,6-trimethyl-phenyl)-amine (0.119 g) and 7-(1-ethyl-propyl)-2-[ethyl-(2,4,6-trimethyl-phenyl)-amino]-4-methyl-7H-pyrrolo[2,3-d]pyrimidine-5,6-dione.

### EXAMPLE 18

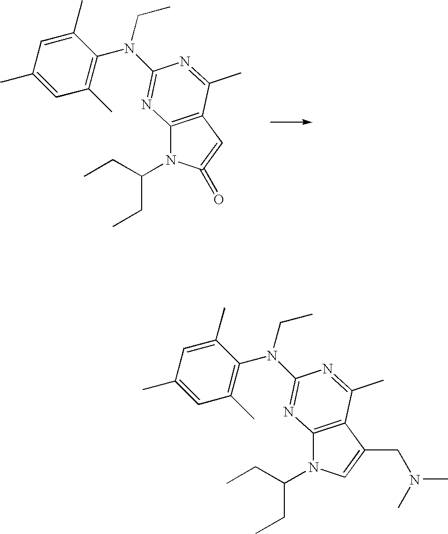

### Synthesis of [5-dimethylaminomethyl-7-(1-ethyl-propyl)-4-methyl-7H-pyrrolo[2,3-d]pyrimidin-2-yl]-ethyl-(2,4,6-trimethyl-phenyl)-amine (1-014)

Formaldehyde, 37wt % solution (0.5 ml) was stirred at room temperature. Dimethylamine in water was added and the reaction mixture was stirred for 15 minutes. Ethyl-[7-(1-ethyl-propyl)-4-methyl-7H-pyrrolo[2,3-d]pyrimidin-2-yl]-(2,4,6-trimethyl-phenyl)-amine (0.05 g) in methanol (0.5 ml) was added and the reaction mixture was stirred at 60° C. for 3 h. Water was added and the product was extracted with dichloromethane. The organic layer was dried over magnesium sulfate, filtered and concentrated under reduced pressure. The residue was purified by a reversed phase column chromatography (eluent: ammonium acetate/acetonitrile) to give [5-dimethylaminomethyl-7-(1-ethyl-propyl)-4-methyl-7H-pyrrolo[2,3-d]pyrimidin-2-yl]-ethyl-(2,4,6-trimethyl-phenyl)-amine (0.015 g).

Tables 1-6 list the compounds obtained in Examples 1-20 and compounds obtained by the similar procedure as in Examples 1-20.

TABLE 1*¹

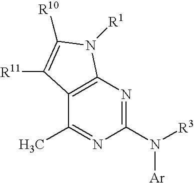

| Com. No. | Ex. No. | 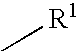 | R³ | R¹⁰ | R¹¹ | 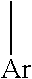 | MS | R.T. (min |
|---|---|---|---|---|---|---|---|---|
| 1-001 | 3 | 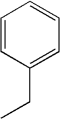 | Et | H | H | 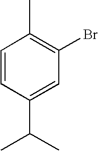 | ESI 463 (M⁺+1) | 14.0 |
| 1-002 | 3 | 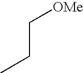 | Et | H | H | 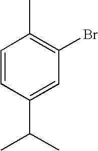 | ESI 431 (M⁺+1) | 7.3 |
| 1-003 | 2 | 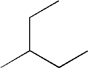 | Et | H | H | 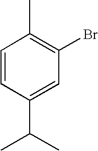 | EI 442 (M⁺) | 19.4 |
| 1-004 | 2 | 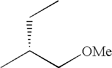 | Et | H | H | 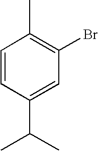 | ESI 481 (M⁺+Na) | 12.4 |
| 1-005 | 3 | 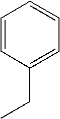 | Et | H | H | 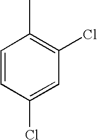 | ESI 411 (M⁺+1) | 9.9 |
| 1-006 | 3 | 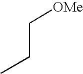 | Et | H | H | 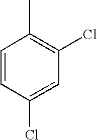 | EI 378 (M⁺) | 6.0 |
| 1-007 | 2 | 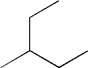 | Et | H | H | 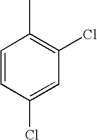 | EI 390 M⁺) | 14.9 |
| 1-008 | 17 | 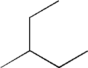 | Et | H | H | 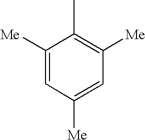 | ESI 365 (M⁺+1) | 19.2 |
| 1-009 | 1 | 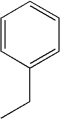 | H | H | H | 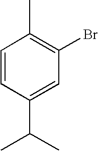 | ESI 435 (M⁺+1) | 11.0 |
| 1-010 | 1 | 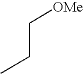 | H | H | H | 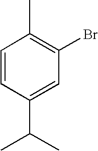 | ESI 403 (M⁺+1) | 6.2 |
| 1-011 | 1 | 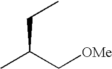 | Et | H | H | 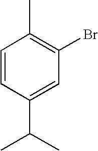 | ESI 481 (M⁺+Na) | 11.8 |
| 1-012 | 1 | 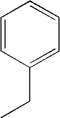 | H | H | H | 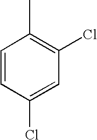 | ESI 383 (M⁺+1) | 8.3 |
| 1-013 | 1 | 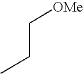 | H | H | H | 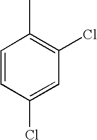 | EI 350 (M⁺) | 5.2 |
| 1-014 | 18 | 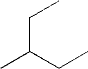 | Et | H | CH₂NMe₂ | 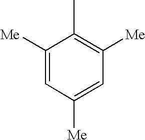 | ESI 444 (M⁺+Na) | 10.2 |
| 1-015 | 8 | 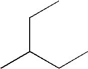 | Et | H | Me | 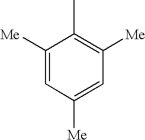 | ESI 401 (M⁺+Na) | 20.5 |

*¹Com. No. = compound number, Ex. No. = example number, MS = mass spectrum, ESI = electrospray ionization, EI = electron ionization, Me = methyl, Et = ethyl, R.T. = retention time on HPLC, HPLC conditions: Capcell Pak UG120, 4.6 mm × 150 mm, Shiseido; Flow rate: 1.0 ml/min; mobile phase: acetonitrile/0.05 M ammomium acetate aqueous solution (80:20), pH of the solvent was adjusted to 7.4 with aqueous ammonia or acetic acid.

TABLE 2*¹

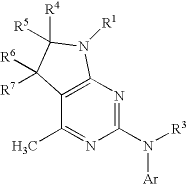

| Com. No. | Ex. No. | 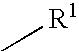 | R³ | R⁴ | R⁵ | R⁶ | R⁷ | 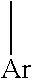 | MS | R.T. (min |
|---|---|---|---|---|---|---|---|---|---|---|
| 2-001 | 8 | 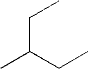 | Et | H | H | Me | H | 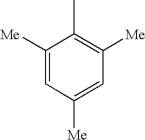 | ESI 381 (M⁺+1) | 3.6 |

*¹Com. No. = compound number, Ex. No. example number, Me = methyl, Et = ethyl, MS = mass spectrum, ESI = electrospray ionization, EI = electron ionization, R.T. = retention time on HPLC, HPLC conditions: Capcell Pak UG120, 4.6 mm × 150 mm, Shiseido; Flow rate: 1.0 ml/min; mobile phase: acetonitrile/0.05M ammonium acetate aqueous solution (80:20), pH of the solvent was adjusted to 7.4 with aqueous ammonia or acetic acid.

TABLE 3*¹

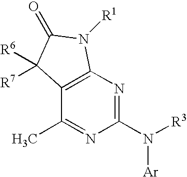

| Com. Ex. | Ex. No. | 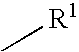 | R³ | R⁶ | R⁷ | 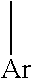 | MS | R.T. (min |
|---|---|---|---|---|---|---|---|---|
| 3-001 | 6 | 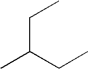 | Et | H | H | 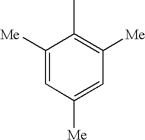 | EI 380 (M⁺) | 9.9 |
| 3-002*² | 10 | 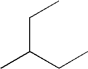 | Et | 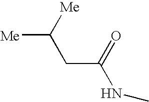 | H | 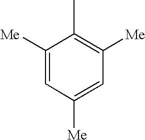 | ESI 480 (M⁺+1) | 4.6 |
| 3-003*² | 10 | 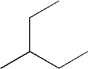 | Et | 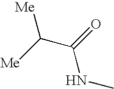 | H | 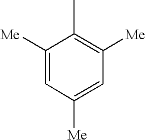 | ESI 466 (M⁺+1) | 4.4 |
| 3-004*² | 10 | 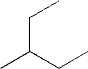 | Et | 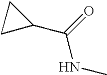 | H | 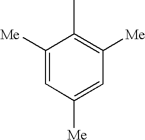 | ESI 464 (M⁺+1 | ) 4.3 |
| 3-005*² | 10 | 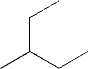 | Et | 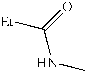 | H | 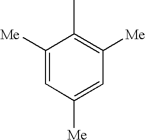 | ESI 452 (M⁺+1) | 4.3 |
| 3-006*² | 10 | 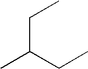 | Et | 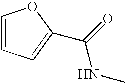 | H | 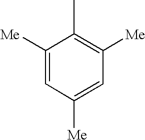 | ESI 490 (M⁺+1) | 4.2 |
| 3-007 | 11 | 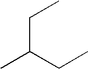 | Et | 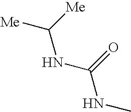 | H | 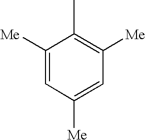 | ESI 503 (M⁺+Na) | 5.9 |
| 3-008 | 11 | 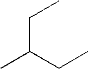 | Et | 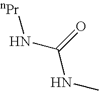 | H | 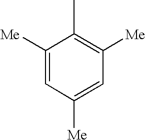 | ESI 503 (M⁺+Na) | 5.9 |
| 3-009 | 13 | 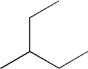 | Et | Me | Me | 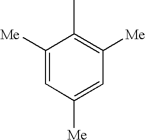 | EI 408 (M⁺+1) | 17.1 |
| 3-010 | 12 | 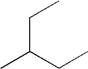 | Et | 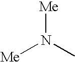 | H | 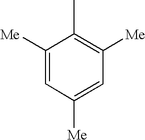 | EI 423 (M⁺) | 17.4 |
| 3-011 | 10 | 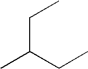 | Et | 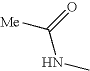 | H | 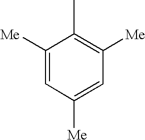 | ESI 460 (M⁺+Na) | 5.5 |
| 3-012 | 7 | 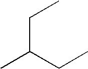 | Et | OH | Me | 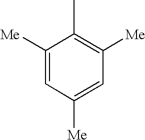 | ESI 433 (M⁺+Na) | 7.6 |
| 3-013 | 7 | 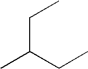 | Et | OH | 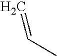 | 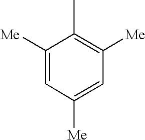 | ESI 445 (M⁺+Na) | 8.5 |
| 3-014 | 7 | 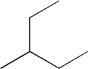 | Et | OH | 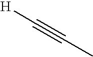 | 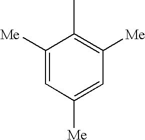 | ESI 443 (M⁺+Na) | 7.9 |
| 3-015 | 7 | 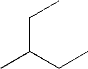 | Et | OH | 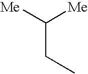 | 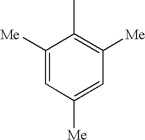 | ESI 475 (M⁺+Na) | 12.4 |
| 3-016 | 7 | 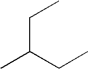 | Et | OH | 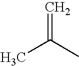 | 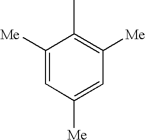 | ESI 459 (M⁺+Na) | 10.7 |
| 3-017 | 7 | 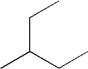 | Et | OH | 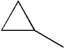 | 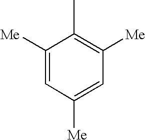 | ESI 459 (M⁺+Na) | 9.3 |
| 3-018 | 14 | 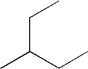 | Et | Et | Et | 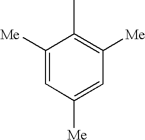 | ESI 437 (M⁺+1) | 24.2 |
| 3-019 | 14 | 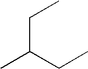 | Et | 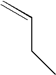 | 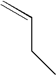 | 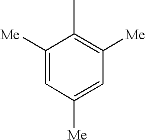 | ESI 483 (M⁺+Na) | 23.7 |
| 3-020 | 7 | 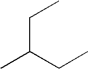 | Et | OH | Et | 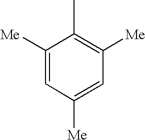 | ESI 447 (M⁺+Na) | 8.7 |
| 3-021 | 14 | 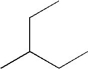 | Et | —CH₂CH₂— | 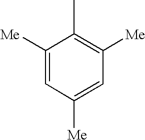 | ESI 429 (M⁺+Na) | 21.6 | |
| 3-022 | 16 | 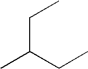 | Et | 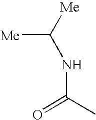 | H | 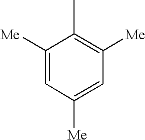 | ESI 488 (M⁺+Na) | 5.8 |

*¹Com. No. = compound number, Ex. No. = example number, Me = methyl, Et = ethyl, MS = mass spectrum, ESI = electrospray ionization, EI = electron ionization, R.T. = retention time on HPLC, HPLC conditions: Capcell Pak UG120, 4.6 mm × 150 mm, Shiseido; Flow rate: 1.0 ml/mm; mobile phase: acetonitrile/0.05M ammonium acetate aqueous solution (80:20), pH of the solvent was adjusted to 7.4 with aqueous ammonia or acetic acid.
*²HPLC conditions: X Terra MS C18 2.5 μm, 4.6 mm × 50 mm; Waters; Flow rate: 1.2 ml/mm; mobile phase: A = 0.5% ammonium acetate in H₂O/CH₃CN (90/10); B = methanol; C = acetonitrile; gradient: start: 90% A + 10% B; end: 10% A + 90% C

TABLE 4*¹

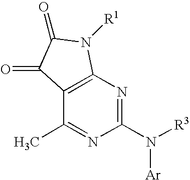

| Com. No. | Ex. No. | 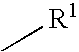 | R³ | 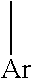 | MS | R.T. (min) |
|---|---|---|---|---|---|---|
| 4-001 | 5 | 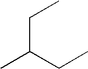 | Et | 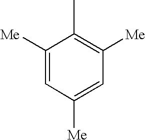 | ESI 417 (M⁺+Na) | 7.9, 9.6 |
| 4-002 | 4 | 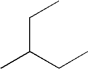 | H | 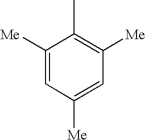 | ESI 389 (M⁺+Na) | 4.1 |

*¹Com. No. = compound number, Ex. No. = example number, Me = methyl, Et = ethyl, MS = mass spectrum, ESI = electrospray ionization, R.T. = retention time on HPLC, HPLC conditions: Capcell Pak UG120, 4.6 mm × 150 mm, Shiseido; Flow rate: 1.0 ml/min; mobile phase: acetonitrile/0.05M ammonium acetate aqueous solution (80:20), pH of the solvent was adjusted to 7.4 with aqueous ammonia or acetic acid.

TABLE 5*¹

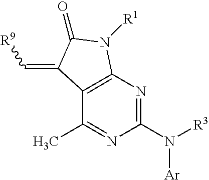

| Com. No. | Ex. No. | 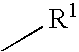 | R³ | 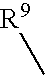 | 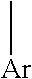 | MS | R.T. (min) |
|---|---|---|---|---|---|---|---|
| 5-001 | 15 | 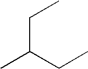 | Et | 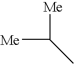 | 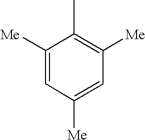 | ESI 457 (M⁺+Na) | 31.8, 42.2 |
| 5-002 | 15 | 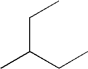 | Et | 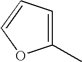 | 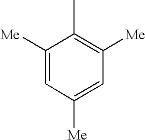 | ESI 481 (M⁺+Na) | 21.6, 38.1 |
| 5-003 | 15 | 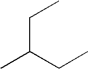 | Et | 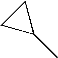 | 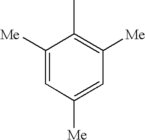 | ESI 455 (M⁺+Na) | 23.5, 26.2 |
| 5-004 | 15 | 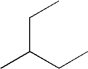 | Et | 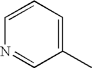 | 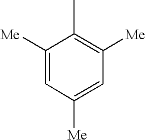 | ESI 492 (M⁺+Na) | 13.1, 16.7 |
| 5-005 | 15 | 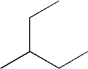 | Et | 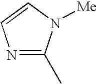 | 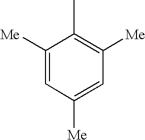 | ESI 495 (M⁺+Na) | 7.4, 9.4 |

*¹Com. No. = compound number, Ex. No. = example number, Me = methyl, Et = ethyl, MS = mass spectrum, ESI = electrospray ionization, R.T. = retention time on HPLC, HPLC conditions: Capcell Pak UG120, 4.6 mm × 150 mm, Shiseido; Flow rate: 1.0 ml/min; mobile phase: acetonitrile/0.05M ammonium acetate aqueous solution (80:20), pH of the solvent was adjusted to 7.4 with aqueous ammonia or acetic acid.

TABLE 6*¹

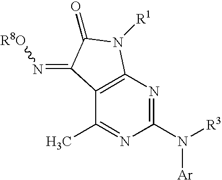

| Com. No. | Ex. No. | 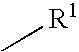 | R³ | R⁸ | 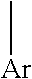 | MS | R.T. (min) |
|---|---|---|---|---|---|---|---|
| 6-001 | 9 | 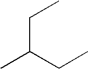 | Et | H | 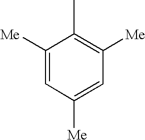 | ESI 432 (M⁺+Na) | 7.8, 10.0 |

*¹Com. No. = compound number, Ex. No. = example number, Me = methyl, Et = ethyl, MS = mass spectrum, ESI = electrospray ionization, R.T. = retention time on HPLC, HPLC conditions: Capcell Pak UG120, 4.6 mm × 150 mm, Shiseido; Flow rate: 1.0 ml/min; mobile phase: acetonitrile/0.05M ammonium acetate aqueous solution (80:20), pH of the solvent was adjusted to 7.4 with aqueous ammonia or acetic acid.

### TEST EXAMPLE

### [CRF Receptor Binding Test]

Monkey amygdala membranes were used as a receptor preparation.

¹²⁵I-CRF was used as ¹²⁵I-labeled ligand.

Binding reaction using the ¹²⁵I-labeled ligand was carried out by the following method described in The Journal of Neuroscience, 7, 88 (1987).

Preparation of Receptor Membranes:

Monkey amygdala was homogenized in 50 mM Tris-HCl buffer (pH 7.0) containing 10 mM MgCl₂, 2 mM EDTA and centrifuged at 48,000×g for 20 min, and the precipitate was washed once with Tris-HCl buffer. The washed precipitate was suspended in 50 mM Tris-HCl buffer (pH 7.0) containing 10 mM MgCl₂, 2 mM EDTA, 0.1% bovine serum albumin and 100 kallilrein units/ml aprotinin, to obtain a membrane preparation.

CRF Receptor Binding Test

The membrane preparation (0.3 mg protein/ml), ¹²⁵I-CRF (0.2 nM) and a test drug were reacted at 25° C. for 2 h. After completion of the reaction, the reaction mixture was filtered by suction through a glass filter (GF/C) treated with 0.3% polyethylene imine, and the glass filter was washed three times with phosphate-buffered saline containing 0.01% Triton X-100. After the washing, the radioactivity of the filter paper was measured in a gamma counter.

The amount of ¹²⁵I-CRF bound when the reaction was carried out in the presence of 1 μM CRF was taken as the degree of nonspecific binding of ¹²⁵I-CRF, and the difference between the total degree of ¹²⁵I-CRF binding and the degree of nonspecific ¹²⁵I-CRF binding was taken as the degree of specific ¹²⁵I-CRF binding. An inhibition curve was obtained by reacting a definite concentration (0.2 nM) of ¹²⁵I-CRF with various concentrations of each test drug under the conditions described above. A concentration of the test drug at which binding of ¹²⁵I-CRF is inhibited by 50% (IC₅₀) was determined from the inhibition curve.

As a result, it was found that compounds 1-003, 1-004, 1-008 and 1-011 can be exemplified as typical compounds having an IC₅₀ value of 200 nM or less.

### ADVANTAGEOUS EFFECT OF THE INVENTION

According to the present invention, compounds having a high affinity for CRF receptors have been provided. These compounds are effective against diseases in which CRF is considered to be involved, such as depression, anxiety, Alzheimer's disease, Parkinson's disease, Huntington's chorea, eating disorder, hypertension, gastro-intestinal diseases, drug dependence, cerebral infarction, cerebral ischemia, cerebral edema, cephalic external wound, inflammation, immunity-related diseases, alpecia, irritable bowel syndrome, sleep disorders, epilepsy, dermatitides, schizophrenia, pain, etc.

1. A pyrrolopyrimidine derivative represented by the following formula [I]: 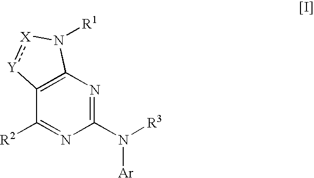
(wherein R¹ is C₁₋₉alkyl, C₂₋₉alkenyl, C₃₋₇cycloalkyl, C₃₋₇cycloalkyl-C₁₋₉alkyl, di(C₃₋₇cycloalkyl)-C₁₋₉alkyl, C₁₋₆alkoxy-C₁₋₉alkyl, di(C₁₋₆alkoxy)-C₁₋₉alkyl, hydroxy-C₁₋₉alkyl, cyano-C₁₋₉alkyl, carbamoyl-C₁₋₉alkyl, di(C₁₋₆alkyl)amino-C₁₋₉alkyl, aryl, heteroaryl, aryl-C₁₋₉alkyl or heteroaryl-C₁₋₉alkyl, in which said aryl and heteroaryl are optionally substituted with one to three substituents independently selected from the group consisting of C₁₋₆alkyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, halogen, C₁₋₆haloalkyl, cyano, nitro, —NR^{1a}R^{1b}, where R^{1a} and R^{1b} are each independently selected from the group consisting of hydrogen, C₁₋₆alkyl and C₁₋₆alkylcarbonyl;
R² is C₁₋₆alkyl or C₁₋₆haloalkyl;
R³ is hydrogen, C₁₋₆alkyl, C₂₋₆alkenyl, C₂₋₆alkynyl, C₃₋₇cycloalkyl, C₃₋₇cycloalkyl-C₁₋₆alkyl, benzyl;
the bond between X and Y is a single bond or a double bond;
wherein (1) when the bond between X and Y is a single bond, X is CR⁴R⁵ or C═O; Y is CR⁶R⁷, C═O, C═N—OR⁸ or C═CH—R⁹; (2) when the bond between X and Y is a double bond, X is CR¹⁰; Y is CR¹¹;
R⁴ and R⁵ are the same or different, and independently are hydrogen or C₁₋₆alkyl;
R⁶ and R⁷ are the same or different, and independently are hydrogen, C₁₋₆alkyl, C₃₋₆cycloalkyl, C₂₋₆alkenyl, C₂₋₆alkynyl, hydroxy, C₁₋₆alkylamino, di(C₁₋₆alkyl)amino, di(C₁₋₆alkyl)amino-C₁₋₆alkyl, C₁₋₆alkylcarbonylamino, C₃₋₆cycloalkylcarbonylamino, arylcarbonylamino, heteroarylcarbonylamino, C₁₋₆alkylaminocarbonyl or C₁₋₆alkylaminocarbonylamino; or R⁶ and R⁷ are taken together to form C₃₋₆cycloalkyl, with the proviso that not both of CR⁴R⁵ and CR⁶R⁷ are CH₂;
R⁸ is hydrogen or C₁₋₆alkyl;
R⁹ is C₁₋₆alkyl, C₃₋₆cycloalkyl, aryl or heteroaryl, wherein said aryl and heteroaryl are optionally substituted with one to three substituents independently selected from the group consisting of halogen or C₁₋₆alkyl;
R¹⁰ is hydrogen or C₁₋₆alkyl;
R¹¹ is hydrogen, C₁₋₆alkyl or di(C₁₋₆alkyl)amino-C₁₋₆alkyl;
Ar is aryl or heteroaryl which aryl or heteroaryl is unsubstituted or substituted with 1 or more substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, cyano, C₁₋₆haloalkyl, trifluoromethoxy, difluoromethoxy, fluoromethoxy and —N(R¹²)R¹³, wherein R¹² and R¹³ are the same or different, and independently are hydrogen or C₁₋₆alkyl), individual isomers thereof or racemic or non-racemic mixtures of isomers thereof, or pharmaceutically acceptable salts and hydrates thereof.

2. The pyrrolopyrimidine derivative according to claim 1 represented by the following formula [II]: 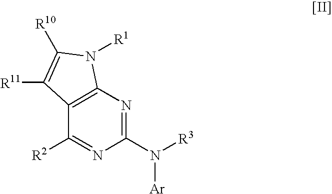
(wherein R¹ is C₁₋₉alkyl, C₂₋₉alkenyl, C₃₋₇cycloalkyl, C₃₋₇cycloalkyl-C₁₋₉alkyl, di(C₃₋₇cycloalkyl)-C₁₋₉alkyl, C₁₋₆alkoxy-C₁₋₉alkyl, di(C₁₋₆alkoxy)-C₁₋₉alkyl, hydroxy-C₁₋₉alkyl, cyano-C₁₋₉alkyl, carbamoyl-C₁₋₉alkyl, di(C₁₋₆alkyl)amino-C₁₋₉alkyl, aryl, heteroaryl, aryl-C₁₋₉alkyl or heteroaryl-C₁₋₉alkyl, in which said aryl and heteroaryl optionally substituted with one to three substituents independently selected from the group consisting of C₁₋₆alkyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, halogen, C₁₋₆haloalkyl, cyano, nitro, —NR^{1a}R^{1b}, where R^{1a} and R^{1b} are each independently selected from the group consisting of hydrogen, C₁₋₆alkyl and C₁₋₆alkylcarbonyl;
R² is C₁₋₆alkyl or C₁₋₆haloalkyl;
R³ is hydrogen, C₁₋₆alkyl, C₂₋₆alkenyl, C₂₋₆alkynyl, C₃₋₇cycloalkyl, C₃₋₇cycloalkyl-C₁₋₆alkyl, benzyl;
R¹⁰ is hydrogen or C₁₋₆alkyl;
R¹¹ is hydrogen, C₁₋₆alkyl or di(C₁₋₆alkyl)amino-C₁₋₆alkyl;
Ar is aryl or heteroaryl which aryl or heteroaryl is unsubstituted or substituted with 1 or more substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, C₁₋₆alkylsulfonyl, aminosulfonyl, mono(C₁₋₆alkyl)aminosulfonyl, di(C₁₋₆alkyl)aminosulfonyl, cyano, haloC₁₋₆alkyl, trifluoromethoxy, difluoromethoxy, fluoromethoxy and —N(R¹²)R¹³, wherein R¹² and R¹³ are the same or different, and independently are hydrogen or C₁₋₆alkyl), individual isomers thereof or racemic or non-racemic mixtures of isomers thereof, or pharmaceutically acceptable salts and hydrates thereof.

3. The pyrrolopyrimidine derivative according to claim 2 represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl, hydroxy-C₁₋₆alkyl, cyano-C₁₋₆alkyl, carbamoyl-C₁₋₆alkyl, di(C₁₋₆alkyl)amino-C₁₋₆alkyl, aryl-C₁₋₆alkyl or heteroaryl-C₁₋₆alkyl; R² is C₁₋₆alkyl; R³ is hydrogen or C₁₋₆alkyl; R¹⁰ is hydrogen or C₁₋₆alkyl; R¹¹ is hydrogen, C₁₋₆alkyl or di(C₁₋₆alkyl)aminoC₁₋₆alkyl; Ar is aryl or heteroaryl which aryl or heteroaryl is unsubstituted or substituted with one to three substituents, which are the same or different, selected from the group consisting of halogen, C₁₋₆alkyl, C₃₋₇cycloalkyl, C₂₋₆alkenyl, C₂₋₆alkynyl, C₁₋₆alkoxy, C₁₋₆alkylthio, cyano, trifluoromethyl, trifluoromethoxy, difluoromethoxy, fluoromethoxy and —N(R¹²)R¹³, wherein R¹² and R¹³ are the same or different, and independently are hydrogen or C₁₋₆alkyl, individual isomers thereof or racemic or non-racemic mixtures of isomers thereof, or pharmaceutically acceptable salts and hydrates thereof.

4. The pyrrolopyrimidine derivative according to claim 2 represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl or aryl-C₁₋₆alkyl; R² is C₁₋₆alkyl; R³ is hydrogen or C₁₋₆alkyl; R¹⁰ is hydrogen or C₁₋₆alkyl; R¹¹ is hydrogen or C₁₋₆alkyl; Ar is phenyl which phenyl is unsubstituted or substituted with one to three substituents, which are the same or different, selected from the group consisting of halogen, C₁₋₃alkyl, C₁₋₃alkoxy, C₁₋₃alkylthio, trifluoromethyl and —N(R¹²)R¹³, wherein R¹² and R¹³ are the same or different, and independently are hydrogen or C₁₋₃alkyl, individual isomers thereof or racemic or non-racemic mixtures of isomers thereof, or pharmaceutically acceptable salts and hydrates thereof.

5. The pyrrolopyrimidine derivative according to claim 2 represented by the formula [II], wherein R¹ is C₁₋₉alkyl, C₃₋₇cycloalkyl, C₃₋₇cycloalkyl-C₁₋₆alkyl, di(C₃₋₇cycloalkyl)-C₁₋₆alkyl, C₁₋₆alkoxy-C₁₋₆alkyl, di(C₁₋₆alkoxy)-C₁₋₆alkyl or aryl-C₁₋₆alkyl; R² is C₁₋₃alkyl; R³ is C₁₋₃alkyl; R¹⁰ is hydrogen; R¹¹ is hydrogen; Ar is phenyl which phenyl is substituted with 2 or 3 substituents, which are the same or different, selected from the group consisting of halogen or C₁₋₃alkyl, individual isomers thereof or racemic or non-racemic mixtures of isomers thereof, or pharmaceutically acceptable salts and hydrates thereof.

6. An antagonist for CRF receptors, comprising a pyrrolopyrimidine derivative, a pharmaceutically acceptable salt thereof or its hydrate according to any one of claims 1 to 5, as an active ingredient.

7. Use of a pyrrolopyrimidine derivative, a pharmaceutically acceptable salt thereof or its hydrate according to any one of claim 1 to 5, for the manufacture of an antagonist for CRF receptors.
